(19) United States
(12) Patent Application Publication
(10) Pub. No.: US 2023/0375534 A1
(43) Pub. Date: Nov. 23, 2023

(54) MEANS AND METHODS TO DIAGNOSE GUT FLORA DYSBIOSIS AND INFLAMMATION

(71) Applicants: VIB VZW, Gent (BE); KATHOLIEKE UNIVERSITEIT LEUVEN, K.U.LEUVEN R&D, Leuven (BE); THE UNIVERSITY OF BRISTOL, Birstol (GB)

(72) Inventors: Jeroen Raes, Berchem (BE); Sebastian Proost, Herent (BE); Gwen Falony, Nossegam (BE); Sara Manuel Araujo Vieira Da Silva, Leuven (BE); Nicholas John Timpson, Bristol (GB); David Allen Hughes, Bristol (GB)

(21) Appl. No.: 18/247,865

(22) PCT Filed: Oct. 5, 2021
(86) PCT No.: PCT/EP2021/077386

§ 371 Date: Apr. 4, 2023

(30) Foreign Application Priority Data
Oct. 5, 2020 (EP) 20200118.6

(51) Int. Cl. G01N 33/53

(52) U.S. Cl. CPC G01N 33/5308; G01N 2800/06

(57) ABSTRACT

The disclosure relates to the field of the human gut microbiome, more particularly, to its effect on health and disease. Provided herein are means and methods to diagnose and treat or reduce the severity of gut flora dysbiosis as well as of gastro-intestinal inflammation and inflammation-associated disorders or conditions in a subject in need thereof.

### CROSS-REFERENCE TO RELATED APPLICATIONS

This application is a national phase entry under 35 U.S.C. § 371 of International Patent Application PCT/EP2021/077386, filed Oct. 5, 2021, designating the United States of America and published as International Patent Publication WO 2022/073973 A1 on Apr. 14, 2022, which claims the benefit under Article 8 of the Patent Cooperation Treaty to European Patent Application Serial No. 202001186, filed Oct. 5, 2020.

### TECHNICAL FIELD

The disclosure relates to the field of the human gut microbiome, more particularly to its effect on health and disease. Provided herein are means and methods to diagnose and treat or reduce the severity of gut flora dysbiosis as well as of gastro-intestinal inflammation and inflammation-associated disorders or conditions in a subject in need thereof.

### BACKGROUND

The human gut is the natural habitat for a large and dynamic bacterial community. These human digestive-tract associated microbes are referred to as the gut microbiome. The human gut microbiome and its role in both health and disease has been the subject of extensive research. Imbalance of the normal gut microbiota—or gut flora dysbiosis—has been linked with gastrointestinal conditions such as inflammatory bowel disease (IBD) and irritable bowel syndrome (IBS), and wider systemic manifestations of disease such as obesity, diabetes, depression and atopy. A problem in mapping the gut microbiome is that the majority of bacteria living in the gut cannot be identified by traditional culturing methods. Therefore, culturing-independent methods have been developed such as 16S rRNA gene sequencing and shotgun sequencing. Based on the overall microbiota composition of a stool sample, bioinformatics analyses such as Dirichlet Multinomial Mixtures (DMM) modeling allow classifying the human gut microbiome in genera-driven clusters or enterotypes (Arumugam et al. 2011 Nature 473: 174-180; Falony et al. 2016 Science 352: 560-564). DMM community typing allows to distinguish a Ruminococcaceae (Rum or R), Prevotella (Prey or P), Bacteroidesl (Bact1 or B1), and Bacteroides2 (Bact2 or B2) enterotype. The latter is a recently described intestinal microbiota configuration embodying gut flora dysbiosis. It is also demonstrated that Bacteroides2 is associated with systemic inflammation, inflammatory bowel disease, primary sclerosing cholangitis, obesity, depression, multiple sclerosis and has a high prevalence in loose stools in humans (Vandeputte et al. 2017 Nature 551: 507-511; Valles-Colomer et al. 2019 Nat. Microbiol. 4: 623-632; Veira-Silva et al. 2019 Nat. Microbiol. 4: 1826-1831; Veira-Silva et al. 2020 Nature 581: 310-315; Reynders et al. 2020 Ann. Clin. Transl. Neur. 7: 406-419). B2 is characterized by a high proportion of Bacteroides, a low proportion of Faecalibacterium and low microbial cell densities (Vandeputte et al. 2017 Nature 551: 507-511). Its prevalence varies from 13% in a general population cohort to as high as 78% in patients with inflammatory bowel disease.

Given the negative correlation between the B2 enterotype and health and given the complexity of B2 enterotype classification (i.e., combining microbiome profiling and flow cytometric enumeration of microbial cells), it would be advantageous to develop an easy and cheap diagnostic preferably based on conventional biological samples for diagnostic purposes such as blood.

### BRIEF SUMMARY

Previously, it was found that the Bacteroides2 enterotype represents gut flora dysbiosis and that it is predominantly present in patients with systemic and intestinal inflammation, indicating that the Bacteroides2 enterotype depicts a vulnerable microbial community associated with disease or pre-disease status. Diagnosing B2 in an early stage would thus be advantageous in order to therapeutically interfere before severe clinical complaints arise. A narrow set of metabolites were identified that allows predicting the B2 enterotype based on the blood serum metabolomics.

Provided is a method of detecting or diagnosing gut flora dysbiosis and/or inflammation in a subject, the method comprising the steps of:
measuring in a biological sample of the subject the level of at least one metabolic biomarker selected from Table 1;
comparing the measured level of the at least one biomarker of the subject sample to that of a control sample; and
determining that the subject suffers from gut flora dysbiosis and/or inflammation if the measured level of the at least one biomarker in the subject sample is increased or decreased relative to the level of that of the control sample and/or if the difference in the measured level of the at least one biomarker in the subject sample is statistically different from that of the control sample.

In particular embodiments, inflammation can be gut inflammation associated with, for example, Crohn's disease, irritable bowel syndrome, inflammatory bowel disease, ulcerative colitis or celiac disease, but inflammation can also be not related to the gut, for example, primary sclerosing cholangitis, spondyloarthritis or multiple sclerosis. The same method steps can also be used for methods of detecting diabetes type 2 or depression in a subject. In other embodiments, the inflammatory disorder is characterized by a TH1, TH17, TH2 and/or TH9 response.

In one embodiment, the biological sample is selected from the list consisting of blood, serum and plasma.

In a particular embodiment, the at least one metabolic biomarker selected from Table 1 is 1H-indole-7-acetic acid, 3-phenylpropionate or cinnamoylglycine. The at least one metabolic biomarker can also be a group of biomarkers. In a further particular embodiment, the group of biomarkers comprises or consists of at least two biomarkers selected from Table 6, at least three biomarkers selected from Table 7, or at least four biomarkers selected from Table 8. The group of biomarkers can also comprise 1H-indole-7-acetic acid, 3-phenylpropionate or cinnamoylglycine and one or more metabolic biomarkers selected from Table 1, Table 6, Table 7, Table 8 and/or Table 9. In a most particular embodiment, the group of biomarkers comprises or consists of the group of biomarkers listed in Table 9, Table 10 or Table 1.

Given that the application discloses single metabolites or sets of metabolites for the purpose of diagnosing gut flora dysbiosis or the B2 enterotype and hence all diseases or disorders that are linked to B2, the application also provides biomarker panels. In one embodiment, these biomarker panels comprise at least two biomarkers selected from Table 6, at least three biomarkers selected from Table 7 or at least four biomarkers selected from Table 8 or comprise 1H-indole-7-acetic acid, 3-phenylpropionate or cinnamoylglycine and at least one biomarker selected from Table 1, 6, 7, 8 or 9. In a most particular embodiment, a biomarker panel is provided comprising or consisting of the group of biomarkers listed in Table 9 or Table 1.

These biomarker panels are also provided for use in diagnosing gut flora dysbiosis, an inflammatory disorder, obesity, diabetes type 2 or depression in a subject, wherein the inflammatory disorder can be selected from the list consisting of spondyloarthritis, ankylosing spondylitis, reactive arthritis, psoriatic arthritis, enteropathic arthritis, undifferentiated spondyloarthritis, juvenile idiopathic arthritis, primary sclerosing cholangitis, multiple sclerosis and any gut inflammation associated therewith. In particular embodiments, the inflammatory disorder is a gut inflammatory disorder selected from the list consisting of Crohn's disease, irritable bowel syndrome, inflammatory bowel disease, ulcerative colitis and celiac disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the correlations between the various metabolites detected to be important for predicting the B2 enterotype from blood serum metabolomics. Two distinct groups can be observed: one group (the cluster on the bottom right containing phenol sulfate and D-Urobilin among others) that is elevated in participants with the B2 enterotype, and one with metabolites decreased in B2 (the cluster on the top left containing hippurate and catechol sulfate among others). The numbers correspond to the compounds listed in Table 4.

FIG. 2 shows the ROC curve when predicting the B2 enterotype vs non-B2 using an ADABoostClassifier taking all metabolites with significant differences (p<0.05 after Bonferroni correction) into account. The shaded area around the curve is the 95% confidence interval.

FIG. 3 shows the ROC curve when predicting the B2 enterotype vs non-B2 using an ADABoostClassifier taking the top ten (based on feature importance of the ADABoost classifier) metabolites as listed in Table 9 into account. The shaded area around the curve is the 95% confidence interval.

### DETAILED DESCRIPTION

### Definitions

The disclosure will be described with respect to particular embodiments and with reference to certain drawings but the disclosure is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun, e.g., “a” or “an,” “the,” this includes a plural of that noun unless something else is specifically stated.

Furthermore, the terms first, second, third, and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

The following terms or definitions are provided solely to aid in the understanding of the disclosure. Unless specifically defined herein, all terms used herein have the same meaning as they would to one skilled in the art of the disclosure. Practitioners are particularly directed to Michael R. Green and Joseph Sambrook, Molecular Cloning: A Laboratory Manual, 4^{th} ed., Cold Spring Harbor Laboratory Press, Plainsview, New York (2012); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Central in this application is the Bacteroides2, B2 or Bact2 enterotype. The B2 enterotype is an intestinal microbiota configuration that is associated with systemic inflammation and has a high prevalence in loose stools in humans (Vandeputte et al. 2017 Nature 551: 507-511). B2 is characterized by a high proportion of Bacteroides, a low proportion of Faecalibacterium and low microbial cell densities, and its prevalence varies from 13% in a general population cohort to as high as 78% in patients with inflammatory bowel disease (Vandeputte et al. 2017 Nature 551: 507-511). The B2 enterotype represents gut flora dysbiosis.

A “high proportion of Bacteroides” refers to a “high relative fraction of the Bacteroides genus” and is defined herein as an at least 10%, at least 20%, at least 30%, at least 40%, at least 50%, at least 60%, at least 70%, at least 80%, at least 90%, at least 100%, at least 1.5-fold, at least two-fold, at least three-fold, at least five-fold or at least ten-fold higher relative abundance compared to the relative abundance of the Bacteroides genus in the stool sample of a healthy subject. “Bacteroides” as used herein refers to a genus of Gram-negative, obligate anaerobic bacteria. Bacteroides species are normally mutualistic, making up the most substantial portion of the mammalian gastrointestinal flora. The Bacteroides genus belongs to the family of Bacteroidaceae and a non-limiting example of a Bacteroides species is B. fragilis.

A “low proportion of Faecalibacterium” refers to a “low relative fraction of the Faecalibacterium genus” and is defined herein as an at least 10%, at least 20%, at least 30%, at least 40%, at least 50%, at least 60%, at least 70%, at least 80%, at least 90%, at least 100%, at least 1.5-fold, at least two-fold, at least three-fold, at least five-fold or at least ten-fold lower relative abundance compared to the relative abundance of the Faecalibacterium genus in the stool sample of a healthy subject. “Faecalibacterium” as used herein refers to a genus of bacteria of which its sole known species, Faecalibacterium prausnitzii is gram-positive, mesophilic, rod-shaped, anaerobic and is one of the most abundant and important commensal bacteria of the human gut microbiota. It is non-spore forming and non-motile. These Faecalibacterium bacteria produce butyrate and other short-chain fatty acids through the fermentation of dietary fiber.

“Relative fraction” or “relative abundance” as used herein refers to the fraction or abundance of a certain genus with respect to or compared to a plurality of other genera present in the stool sample.

“Low microbial cell densities” or “low microbial cell count” as used herein is a microbial cell count that is at least 10%, at least 20%, at least 30%, at least 40%, at least 50%, at least 60%, at least 70%, at least 80%, at least 90%, at least 100%, at least 1.5-fold, at least two-fold, at least three-fold, at least five-fold or at least ten-fold lower than the microbial count of a stool sample of a healthy subject.

“Cell count” as used herein refers to the sample cell density, in order words how many cells, more particularly microbial cells, are present in the sample, more particularly stool sample. Multiple methods are known by the skilled person to quantify microbial cell count in a stool sample, which is typically presented as cells per gram stool.

“Stool sample” and “fecal sample” are used interchangeably and refer to as a sample or aliquot of the stool or feces of a subject, more particularly a mammal, even more particularly a human being, most particularly a patient. The stool sample as used herein comprises the gut microbiome from a human patient to be diagnosed. As used herein, the term “microflora” refers to the collective bacteria in an ecosystem of a host (e.g., an animal, such as a human) or in a single part of the host's body, e.g., the gut. An equivalent term is “microbiota.” As used herein, the term “microbiome” refers to the totality of bacteria, their genetic elements (genomes) in a defined environment, e.g., within the gut of a host, the latter then being referred to as the “gut microbiome.”

As used herein, the term “patient” or “individual” or “subject” typically denotes humans, but may also encompass reference to non-human animals, preferably warm-blooded animals, more preferably mammals, such as, e.g., non-human primates, rodents, canines, felines, equines, ovines, porcines, and the like.

As used herein, the term “gut” generally comprises the stomach, the colon, the small intestine, the large intestine, cecum and the rectum. In addition, regions of the gut may be subdivided, e.g., the right versus the left side of the colon may have different microflora populations due to the time required for digesting material to move through the colon, and changes in its composition in time. Synonyms of gut include the “gastrointestinal tract,” or possibly the “digestive system,” although the latter is generally also understood to comprise the mouth, esophagus, etc.

The term “gut microbiome composition” is equivalent in wording as “gut microbiome profile” and these wordings are used interchangeably herein. A gut microbiome profile represents the presence, absence or the abundance of one or more of bacterial genera identified in a stool sample. The gut microbiome profile can be determined based on an analysis of amplification products of DNA and/or RNA of the gut microbiota, e.g., based on an analysis of amplification products of genes coding for one or more of small subunit rRNA, etc., and/or based on an analysis of proteins and/or metabolic products present in the biological sample. Gut microbiome profiles may be “compared” by any of a variety of statistical analytic procedures.

In microbiology, “16S sequencing” or “16S” refers to a sequence derived by characterizing the nucleotides that comprise the 16S ribosomal RNA gene(s). The bacterial 16S rRNA is approximately 1500 nucleotides in length and is used in reconstructing the evolutionary relationships and sequence similarity of one bacterial isolate to another using phylogenetic approaches.

For the current application “method to detect an inflammatory disorder” is equivalent to a “method to detect the presence or to assess the risk of developing an inflammatory disease.”

The term “inflammation,” “inflammatory disorder” or “inflammatory disease” refers to complex but to the skilled person well known biological response of body tissues to harmful stimuli, such as pathogens, damaged cells, or irritants. Inflammation is not a synonym for infection though. Infection describes the interaction between the action of microbial invasion and the reaction of the body's inflammatory response—the two components are considered together when discussing an infection, and the word is used to imply a microbial invasive cause for the observed inflammatory reaction. Inflammation on the other hand describes purely the body's immunovascular response, whatever the cause may be. Inflammation is a protective response involving immune cells, blood vessels, and molecular mediators. The function of inflammation is to eliminate the initial cause of cell injury, clear out necrotic cells and tissues damaged from the original insult and the inflammatory process, and to initiate tissue repair. The classical signs of inflammation are heat, pain, redness, swelling, and loss of function. Inflammation is a generic response, and therefore it is considered as a mechanism of innate immunity, as compared to adaptive immunity, which is specific for each pathogen. Inflammation can be classified as either acute or chronic. Acute inflammation is the initial response of the body to harmful stimuli and is achieved by the increased movement of plasma and leukocytes (especially granulocytes) from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation, such as mononuclear cells, and is characterized by simultaneous destruction and healing of the tissue from the inflammatory process.

The term ROC or Receiver Operating Characteristic curve refers to a graphical plot that illustrates the diagnostic ability of a binary classifier system or alternatively phrased a probability curve. The area under the curve (often referred to as simply the AUC) refers then to the probability that a classifier will rank a randomly chosen positive instance higher than a randomly chosen negative one. It thus tells how much the model is capable of distinguishing between classes. The higher the AUC, the better the prediction model is. In the ROC curve the sensitivity or the True Positive Rate (TPR) is plotted against the False Positive Rate or 1-specificity, where TPR is on y-axis and FPR is on the x-axis.

A Metabolic Profile Correlates with the B2 Gut Enterotype

The goal of the study that led to this disclosure was to find a narrow set of metabolites that allows the prediction of the B2 enterotype based on blood serum metabolomics. To this end various machine learning approaches were used to select relevant metabolites. Starting with 1000 different metabolites that were measured in blood samples, it was found that the level of 59 metabolic analytes correlated with the B2 enterotype. A biomarker panel of 59 metabolic analytes depicted in Table 1 provided an excellent B2 prediction. Surprisingly, the list of metabolites could be narrowed down to the ten biomarkers listed in Table 9 and even down to the five biomarkers listed in Table 10 while maintaining a highly reliable prediction (ROC AUC>0.8).

Therefore, in a first aspect a biomarker panel is provided comprising at least four, at least five, at least six, at least seven, at least eight, at least nine, at least ten, at least eleven, at least twelve, at least thirteen, at least fourteen, or at least fifteen metabolic biomarkers selected from Table 1. In one particular embodiment, a biomarker panel is provided comprising at least four, at least five, at least six, at least seven, at least eight or nine metabolic biomarkers selected from Table 9. In another particular embodiment, the biomarker panels comprise at least 3-phenylpropionate, isoursodeoxycholate and/or p-cresol sulfate.

In a most particular embodiment, a biomarker panel is provided comprising or consisting of the group of biomarkers listed in Table 9, Table 10 or Table 1.

TABLE 9
A biomarker panel consisting of a set of ten metabolites
that can be used to generate high quality predictions (ROC
AUC >0.8) for the B2 enterotype. (2) means isoform 2.

| | |
|---|---|
| 3-phenylpropionate (hydrocinnamate) | 5-hydroxyhexanoate |
| isoursodeoxycholate | anthranilate |
| p-cresol sulfate | adenosine 5′-monophosphate |
| | (AMP) |
| indole-3-carboxylic acid | glycoursodeoxycholate |
| glycolithocholate sulfate | glucuronide of C14H22O4 (2) |

TABLE 10
A biomarker panel consisting of a set of ten metabolites
that can be used to generate high quality predictions
(ROC AUC >0.8) for the B2 enterotype.

| | | |
|---|---|---|
| N-acetyl-cadaverine | hippurate | I-urobilinogen |
| phenol sulfate | imidazole propionate | |

In an attempt to restrict the number of metabolic biomarkers to still reliably predict the B2 enterotype, several combinations of two or more metabolites were tested. Surprisingly, acceptable predictions (ROC AUC>0.7) could be obtained from single metabolic markers (i.e., 1H-indole-7-acetic acid, 3-phenylpropionate and cinnamoylglycine), while several combinations of two or more other metabolites were found to be usable to predict the B2 enterotype as well (see Example 3).

Therefore, in yet another embodiment, a biomarker panel is provided comprising or consisting of at least two metabolic biomarkers selected from Table 6.

In another embodiment, a biomarker panel is provided comprising or consisting of at least three metabolic biomarkers selected from Table 7.

In another embodiment, a biomarker panel is provided comprising or consisting of at least four metabolic biomarkers selected from Table 8.

In yet another embodiment, a biomarker panel is provided comprising 1H-indole-7-acetic acid, 3-phenylpropionate or cinnamoylglycine and further comprising at least one additional metabolic biomarker. In a particular embodiment, the at least one additional metabolic biomarker is selected from Table 9, Table 10 or from Table 1. In yet another embodiment, a biomarker panel is provided comprising or consisting of 1H-indole-7-acetic acid, 3-phenylpropionate and/or cinnamoylglycine.

TABLE 6
A biomarker panel consisting of thirteen metabolites.
Combinations of at least two of these metabolites
predict the B2 enterotype with a ROC AUC >0.7.

| | | |
|---|---|---|
| imidazole propionate | 4-hydroxycoumarin | catechol sulfate |
| glycolithocholate sulfate | I-urobilinogen | phenol sulfate |
| isoursodeoxycholate | p-cresol sulfate | hippurate |
| hydroxyhexanoate | N-acetyl-cadaverine | glutarate (C5-DC) |
| 5alpha-androstan-3beta, | | |
| 17alpha-diol disulfate | | |

TABLE 7
A biomarker panel consisting of five metabolites. Combinations
of at least three of these metabolites predict the B2 enterotype
with a ROC AUC >0.7. [2] means isoform 2.

| | |
|---|---|
| ursodeoxycholate | 7-alpha-hydroxy-3-oxo-4-cholestenoate |
| | (7-Hoca) |
| indole-3-carboxylic acid, | palmitoyl-oleoy1-glycerol (16:0/18:1) [2] |
| phenol glucuronide | |

TABLE 8
A biomarker panel consisting of sixteen metabolites. Combinations
of at least four of these metabolites predict the B2 enterotype
with a ROC AUC >0.7. [2] means isoform 2.

| | |
|---|---|
| glucuronide of C14H22O4 [2] | etiocholanolone glucuronide |
| indolepropionylglycine | 1-oleoyl-GPE (18:1) |
| N-acetylglucosamine conjugate | 4-hydroxyphenylacetate |
| of C24H40O4 bile acid | |
| glycoursodeoxycholate | dihydroferulic acid |
| phenylacetate | 1-(1-enyl-palmitoyl)-2-oleoyl-GPC |
| | (P-16:0/18:1) |
| 4-ethylphenylsulfate | adenosine 5′-monophosphate (AMP) |
| glycochenodeoxycholate sulfate | 1-(1-enyl-palmitoyl)-2-palmitoyl-GPC |
| | (P-16:0/16:0) |
| anthranilate | palmitoyl sphingomyelin (d18:1/16:0) |

Several minimal combinations could be identified that rendered an ROC AUC>0.8. For example, this is the case for the combinations of at least six metabolites from Table 11 or of at least eleven from Table 12. Therefore, in yet another embodiment, a biomarker panel is provided comprising or consisting of at least six, at least seven, at least eight, at least nine or at least ten metabolic biomarkers selected from Table 11.

In yet another embodiment, a biomarker panel is provided comprising or consisting of at least eleven, at least twelve, at least thirteen, at least fourteen or at least fifteen metabolic biomarkers selected from Table 12.

TABLE 11
A biomarker panel consisting of a set of fifteen metabolites.
Combination of at least six of these metabolites predict the
B2 enterotype with a ROC AUC >0.8. (2) means isoform 2.

| | | |
|---|---|---|
| p-cresol sulfate | 3-phenylpropionate | anthranilate |
| 4-hydroxycoumarin | glutarate (C5-DC) | indole-3-carboxylic acid |
| glycolithocholate | isoursodeoxycholate | 1H-indole-7-acetic acid |
| sulfate | | |
| glycoursodeoxycholate | glucuronide of | 5-hydroxyhexanoate |
| | C14H22O4 (2) | |
| 5alpha-androstan- | 1-(1-enyl-palmitoyl)- | adenosine 5′- |
| 3beta,17alpha-diol | 2-palmitoyl-GPC | monophosphate (AMP) |
| disulfate | (P-16:0/16:0) | |

TABLE 12
A biomarker panel consisting of a set of twenty-one metabolites.
Combination of at least eleven of these metabolites predict the
B2 enterotype with a ROC AUC >0.8. (2) of [2] means isoform 2.

| catechol sulfate | 1-oleoyl-GPE (18:1) | phenylacetate |
|---|---|---|
| cinnamoylglycine | carotene diol (2) | ursodeoxycholate |
| 2-acetamidophenol | 5-hydroxyindole | etiocholanolone |
| sulfate | sulfate | glucuronide |
| dihydroferulic acid | phenol glucuronide | glucuronide of |
| | | C19H28O4 (2) |
| glycochenodeoxycholate | indolepropionylglycine | 4- |
| sulfate | | hydroxyphenylacetate |
| palmitoyl sphingomyelin | oleoyl-arachidonoyl- | palmitoyl-oleoyl- |
| (d18:1/16:0) | glycerol (18:1/20:4) | glycerol (16:0/18:1) |
| | [2] | [2] |
| N-acetylglucosamine | 1-(1-enyl-palmitoyl)-2- | 7-alpha-hydroxy-3- |
| conjugate of C24H40O4 | oleoy1-GPC (P- | oxo-4-cholestenoate |
| bile acid | 16:0/18:1) | (7-Hoca) |

From here on, the biomarker panels disclosed above will be referred to as “one of the biomarker panels of the application” or as “any of the biomarker panels of the application.”

In a second aspect; any of the biomarker panels of the application is provided for use in diagnosing a disease or disorder. Indeed, the B2 enterotype represent gut flora dysbiosis and is associated with health problems and several inflammatory disorders. People who have this dysbiotic enterotype have a higher blood concentration of C-reactive protein—a hallmark of inflammation—than do individuals who have other enterotypes (Costea et al. 2018 Nat. Microbiol. 3: 8-16). More than 75% of individuals who have IBD have the B2 enterotype in contrast to fewer than 15% of people who do not have the disease (Veira-Silva et al. 2019 Nat. Microbiol. 4: 1826-1831). The B2 enterotype is also correlated to primary sclerosing cholangitis (Veira-Silva et al. 2019 Nat. Microbiol, 4: 1826-1831), multiple sclerosis (Reynders et al. 2020 Ann. Clin. Trans., Neur. 7: 406-419), depression (Valles-Colomer et al. 2019 Nat. Microbiol. 4: 623-632) and obesity (Veira-Silva et al. 2020 Nature. 581: 310-315).

Any of the biomarker panels of the application are also provided for use in detecting in a subject a gut flora microbiome associated with or predictive for a disease or disorder. In one embodiment, the disease or disorder is gut flora dysbiosis and/or an inflammatory disorder in a subject. In another embodiment, the disease or disorder is obesity, diabetes type 2 or depression.

In a particular embodiment, the inflammatory disorder is selected from the list consisting of spondyloarthritis, ankylosing spondylitis, reactive arthritis, psoriatic arthritis, enteropathic arthritis, undifferentiated spondyloarthritis, juvenile idiopathic arthritis, primary sclerosing cholangitis, multiple sclerosis, a gut inflammatory disorder, inflammatory bowel disease (IBD), Crohn's disease (CD), ulcerative colitis (UC), irritable bowel syndrome (IBS), celiac disease and any combination thereof and any gut inflammation associated with one of the above listed inflammatory disorders. In another particular embodiment, the inflammatory disorder is characterized by a TH1, TH17, TH2 and/or TH9 response.

In another aspect, the use of any of the biomarker panels of the application is provided to classify, categorize or distinguish different gut flora microbiomes based on isolated biological samples. The use of any of the biomarker panels of the application is also provided to distinguish a B2 enterotype or a dysbiotic gut microbiome or a gut microbiome associated with gut flora dysbiosis and/or an inflammatory disorder from a non-B2 enterotype or a gut microbiome not associated with gut flora dysbiosis and/or an inflammatory disorder.

In a fourth aspect, methods of detecting a disease or disorder in a subject are provided comprising the following steps:
measuring in a biological sample of the subject the level of at least one metabolic biomarker selected from any of the metabolic biomarker panels of the application;
comparing the measured level of the at least one biomarker of the subject sample to that of a control sample; and
determining that the subject suffers from a disease or disorder if the measured level of the at least one biomarker in the subject sample is increased or decreased relative to the level of the at least one biomarker in the control sample and/or if the difference between the measured level of the at least one biomarker in the subject sample and that of the control sample is statistically significant.

In one embodiment, the disease or disorder is gut flora dysbiosis and/or an inflammatory disorder. In another embodiment, the disease or disorder is obesity, diabetes type 2 or depression. In a particular embodiment, the inflammatory disorder is selected from the list consisting of spondyloarthritis, ankylosing spondylitis, reactive arthritis, psoriatic arthritis, enteropathic arthritis, undifferentiated spondyloarthritis, juvenile idiopathic arthritis, primary sclerosing cholangitis, multiple sclerosis, a gut inflammatory disorder, inflammatory bowel disease (IBD), Crohn's disease (CD), ulcerative colitis (UC), irritable bowel syndrome (IBS), celiac disease and any combination thereof and any gut inflammation associated with one of the above listed inflammatory disorders. In another particular embodiment, the inflammatory disorder is characterized by a TH1, TH17, TH2 and/or TH9 response.

In particular embodiments, the above disclosed methods steps are provided for a method of detecting or diagnosing in a subject a gut microbiome associated with or predictive for gut flora dysbiosis and/or an inflammatory disorder. Even more particular, the methods steps are also provided for methods of distinguishing or predicting or diagnosing different gut flora microbiomes, more particularly a gut flora microbiome associated with gut flora dysbiosis or inflammation, most particularly a Bacteroides2 enterotype.

In one embodiment, the biological sample for the methods of current application is selected from the list consisting of blood, serum and plasma.

In one embodiment, the at least one metabolic biomarker selected from any of the metabolic biomarker panels of the application is 1H-indole-7-acetic acid (CAS No. 39689-63-9), 3-phenylpropionate (CAS No. 501-52-0, alternative names are hydrocinnamate and 3-phenylpropanoate) or cinnamoylglycine (CAS No. 16534-24-0). This is equivalent is saying that the at least one metabolic biomarker is selected from Table 5.

TABLE 5
A biomarker panel consisting of 1H-indole-7-acetic
acid, 3-phenylpropionate and cinnamoylglycine.

| | | |
|---|---|---|
| 1H-indole-7-acetic acid | 3-phenylpropionate | cinnamoylglycine |

In another embodiment, the at least one metabolic biomarker selected from any of the metabolic biomarker panels of the application is a group of metabolic biomarkers. In a more particular embodiment, the group of metabolic biomarkers comprises at least two metabolic biomarkers selected from Table 6, or at least three metabolic biomarkers selected from Table 7 or at least four metabolic biomarkers selected from Table 8 or at least six metabolic markers selected from Table 11 or at least eleven metabolic biomarkers selected from Table 12.

In yet another embodiment, the group of metabolic biomarkers comprises at least one metabolic biomarker selected from the list consisting of 1H-indole-7-acetic acid, 3-phenylpropionate (hydrocinnamate) and cinnamoylglycine and further comprises at least one, at least two, at least three or at least four additional metabolic biomarker(s). In a further particular embodiment, the at least one, at least two, at least three or at least four additional metabolic biomarker(s) is/are selected from Table 6, 7, 8, 9, 10, 11, 12 or 1.

In yet another embodiment, the group of metabolic biomarkers comprises at least four, at least five, at least six, at least seven, at least eight, at least nine, at least ten, at least eleven, at least twelve, at least thirteen, at least fourteen or at least fifteen metabolic biomarkers selected from Table 1. In yet another embodiment, the group of metabolic biomarkers comprises at least four, at least five, at least six, at least seven, at least eight or nine metabolic biomarkers selected from Table 9. In yet another embodiment, the group of metabolic biomarkers comprises at least 3-phenylpropionate, isoursodeoxycholate and/or p-cresol sulfate.

In a most particular embodiment, the group of metabolic biomarkers comprises or consists of the group of biomarkers listed in Table 9, Table 10 or Table 1.

Blood serum metabolite levels can be measured in parallel using Liquid Chromatography paired with Mass Spectrometry (LC-MS), tandem mass-spectrometry (LC-MS/MS), gas chromatography paired with mass spectrometry (GC-MS), high performance liquid chromatography using UV or fluorescent detection, nuclear magnetic resonance (NMR) spectroscopy or combinations thereof. The skilled person is familiar with the measurements, as a plethora of platforms are commercially available to perform these measurements. Alternatively, when considering a limited set of key metabolic markers one or more targeted essays could also be used.

With access to samples and a labeled dataset, where the enterotype is known, a classifier can be trained on (a subset of) the measured metabolites. Any classifier that can predict a class label from one or more continuous features can be used, this includes, but is not limited to Decision Trees, Random Forest Classifiers, Support Vector Classifiers, Stochastic Gradient Decent Classifier and the ADABoostClassifier. Various implementations for these classifiers are available in Scikit-learn (for the python language), Machine Learning for R (mlr library for R), etc. Once trained on a labeled set, metabolite levels from patients' samples with an unknown enterotype can be provided to the trained classifier to obtain a predicted class (in this case B2 or non-B2).

Alternatively, a set of logical rules, depending on upper and lower thresholds for key metabolic markers, could also be designed to characterize the B2 enterotype. Indeed, in the methods described herein, the determination step can be based on an increased or decreased level of the at least one metabolic biomarker in the subject sample compared to that in the control sample (see also Table 1). If 3-phenylpropionate is measured as one of the metabolic biomarkers then a decreased level is predictive for the disease or disorder (e.g., gut flora dysbiosis and/or an inflammatory disorder, obesity, diabetes type 2, depression) or for a gut microbiome associated with or predictive for the disease or disorder. For cinnamoylglycine a decreased level is predictive, for 5-hydroxyhexanoate a decreased level, for 5alpha-androstan-3beta,17alpha-diol disulfate a decreased level, for 4-hydroxycoumarin a decreased level, for hippurate a decreased level, for phenol sulfate an increased level, for glucuronide of C19H28O4 a decreased level, for isoursodeoxycholate an increased level, for imidazole propionate an increased level, for indolepropionylglycine a decreased level, for I-urobilinogen an increased level, for N-acetyl-cadaverine an increased level, for glycoursodeoxycholate an increased level, for D-urobilin an increased level, for 11-ketoetiocholanolone glucuronide a decreased level, for 7-alpha-hydroxy-3-oxo-4-cholestenoate (7-Hoca) an increased level, for glutarate (C5-DC) an increased level, for 1H-indole-7-acetic acid a decreased level, for carotene diol a decreased level, for ursodeoxycholate an increased level, for taurolithocholate 3-sulfate a decreased level, for indole-3-carboxylic acid a decreased level, for palmitoyl-linoleoyl-glycerol (16:0/18:2) an increased level, for N2,N5-diacetylornithine a decreased level, for glycolithocholate sulfate a decreased level, for beta-cryptoxanthin a decreased level, for phenylacetate a decreased level, for 3-(4-hydroxyphenyl)propionate an increased level, for 1-(1-enyl-palmitoyl)-2-palmitoyl-GPC (P-16:0/16:0) a decreased level, for oleoyl-oleoyl-glycerol (18:1/18:1) [2] an increased level, for etiocholanolone glucuronide a decreased level, for palmitoyl-oleoyl-glycerol (16:0/18:1) [2] an increased level, for 1-(1-enyl-palmitoyl)-2-oleoyl-GPC (P-16:0/18:1) a decreased level, for 3-methyladipate a decreased level, for 1-oleoyl-GPE (18:1) an increased level, for palmitoyl sphingomyelin (d18:1/16:0) a decreased level, for carotene diol (2) a decreased level, for oleoyl-arachidonoyl-glycerol (18:1/20:4) [1] an increased level, for p-cresol sulfate a decreased level, for anthranilate a decreased level, for oleoyl-linoleoyl-glycerol (18:1/18:2) [2] an increased level, for guanidinosuccinate a decreased level, for 5-hydroxyindole sulfate a decreased level, for 2-acetamidophenol sulfate a decreased level, for glycosyl-N-tricosanoyl-sphingadienine (d18:2/23:0) a decreased level, for 4-hydroxyglutamate an increased level, for 4-ethylphenylsulfate an decreased level, for adenosine 5′-monophosphate (AMP) a decreased level and for glycochenodeoxycholate sulfate an increased level.

In a further particular embodiment, the differences in level of the measured metabolic biomarker between the subject sample and that of the control sample are statistically significant.

The term “statistically significant” or “statistically significantly” different is well known by the person skilled in the art. Statistical significance plays a pivotal role in statistical hypothesis testing. It is used to determine whether the null hypothesis should be rejected or retained. It states that the results are obtained because of chance and are not supporting a real change or difference between two data sets. The null hypothesis is the default assumption that what one is trying to prove did not happen. In contrast, the alternative hypotheses states that the obtained results support the theory being investigated. For the null hypothesis to be rejected (and thus the alternative hypothesis to be accepted), an observed result has to be statistically significant, i.e., the observed p-value is less than the pre-specified significance level a. The p stands for probability and measures how likely it is that the null hypothesis is incorrectly rejected and thus that any observed difference between data sets is purely due to chance. In most cases the significance level a is set at 0.05.

In one embodiment, the control sample is representative of matched human subjects. In one embodiment, the control sample is a sample from a subject with a non-B2 enterotype or alternatively phrased a subject with a Bacteriodesl, Prevotella or Ruminococcaceae enterotype. In another embodiment, the control sample is a sample from a subject with a gut microbiome that is not associated with or predictive for gut flora dysbiosis and/or inflammatory disorder or obesity or diabetes type 2 or depression. In other embodiments, the control sample is a negative control sample from a healthy individual, i.e., comparable individual not suffering from or diagnosed with gut flora dysbiosis and/or inflammatory disorders or obesity or diabetes type 2 or depression or a comparable individual not having an enterotype or a gut microbiome associated with or predictive for gut flora dysbiosis and/or inflammatory disorders.

The application also provides methods to detect the presence or to assess the risk of developing a disease or disorder, or a gut microbiome associated with or predictive of a disease or disorder in a patient, comprising the steps of:
determining a metabolic profile from a biological sample obtained from the patient and comparing the profile to one or more metabolic reference profiles, wherein the one or more metabolic reference profiles comprise at least one of a positive metabolic reference profile based on results from control subjects with the disease or disorder or with a gut microbiome associated with or predictive of the disease or disorder, and a negative metabolic reference profile based on results from control subjects without the disease or disorder or without a gut microbiome associated with or predictive of the disease or disorder,
if the metabolic profile for the patient statistically significantly matches the positive metabolic reference profile, then concluding that the patient has or is at risk of developing the disease or disorder or of a gut microbiome associated with or predictive of the disease or disorder in a patient; and/or
if the metabolic profile for the patient statistically significantly matches the negative metabolic reference profile, then concluding that the patient does not have or is not at risk of developing the disease or disorder or does not have a gut microbiome associated with or predictive of the disease or disorder in a patient.

In one embodiment, a positive metabolic reference profile is a metabolic reference profile from a subject with a B2 enterotype and a negative metabolic reference profile is a metabolic reference profile from a subject not having a B2 enterotype or alternatively phrased having a B1, R or P enterotype.

In one embodiment, the disease or disorder is gut flora dysbiosis and/or an inflammatory disorder. In another embodiment, the disease or disorder is obesity, diabetes type 2 or depression. In a particular embodiment, the inflammatory disorder is selected from the list consisting of spondyloarthritis, ankylosing spondylitis, reactive arthritis, psoriatic arthritis, enteropathic arthritis, undifferentiated spondyloarthritis, juvenile idiopathic arthritis, primary sclerosing cholangitis, multiple sclerosis, a gut inflammatory disorder, inflammatory bowel disease (IBD), Crohn's disease (CD), ulcerative colitis (UC), irritable bowel syndrome (IBS), celiac disease and any combination thereof and any gut inflammation associated with one of the above listed inflammatory disorders. In another particular embodiment, the inflammatory disorder is characterized by a TH1, TH17, TH2 and/or TH9 response.

In another embodiment, the metabolic profile is determined from a biological sample, which can be blood, serum or plasma. In a more particular embodiment, the biological sample consists of blood, serum and plasma.

In another embodiment, the metabolic profile comprises an indication of the presence and/or abundance of at least one, at least two, at least three, at least four, at least five, at least six, at least seven, at least eight, at least nine, at least ten, at least eleven, at least twelve, at least thirteen, at least fourteen or at least fifteen metabolic biomarkers selected from Table 1. In a more particular embodiment, the metabolic profile comprises an indication of the presence and/or abundance of at least one, at least two, at least three, at least four, at least five, at least six, at least seven, at least eight or nine selected from Table 9.

In another embodiment, the metabolic profile comprises an indication of the presence and/or abundance of 1H-indole-7-acetic acid, 3-phenylpropionate or cinnamoylglycine.

In another embodiment, the metabolic profile comprises an indication of the presence and/or abundance of at least two metabolic biomarkers selected from Table 6, or of at least three metabolic biomarkers selected from Table 7 or of at least four metabolic biomarkers selected from Table 8 or at least six metabolic biomarkers selected from Table 11 or at least eleven metabolic biomarkers selected from Table 12.

In yet another embodiment, the metabolic profile comprises an indication of the presence and/or abundance of at least one metabolic biomarker selected from the list consisting of 1H-indole-7-acetic acid, 3-phenylpropionate and cinnamoylglycine and further at least one, at least two, at least three or at least four additional metabolic biomarker(s). In a further particular embodiment, the at least one, at least two, at least three or at least four additional metabolic biomarker(s) is/are selected from Table 1, 6, 7, 8, 9, 10, 11 or 12.

In another embodiment, the metabolic profile is obtained by one of the metabolic biomarker panels disclosed in current application.

In a most particular embodiment, the metabolic profile comprises an indication of the presence and/or abundance of the biomarkers listed in Table 9, Table 10 or Table 1. With abundance it is meant the quantification of the metabolic biomarkers. The quantification can be absolute quantification or relative quantification compared reference values.

In a fifth aspect, methods of diagnosing and treating an inflammatory disorder in a subject are provided. The methods comprise the steps from the methods provided in the fourth aspect of current application further comprising a step of administering an effective amount of anti-inflammatory drugs to the subject. This is equivalent as saying that methods are provided of diagnosing and treating an inflammatory disorder in a patient, comprising administering anti-inflammatory therapy to the patient if the blood, plasma or serum metabolic profile for the patient statistically significantly matches that of a Bacteroides2 enterotype. In a particular embodiment, the match is performed by using one of the biomarkers from the application.

In further embodiments, the inflammatory disorder is selected from the list consisting of spondyloarthritis, ankylosing spondylitis, reactive arthritis, psoriatic arthritis, enteropathic arthritis, undifferentiated spondyloarthritis, juvenile idiopathic arthritis, primary sclerosing cholangitis, multiple sclerosis, a gut inflammatory disorder, inflammatory bowel disease (IBD), Crohn's disease (CD), ulcerative colitis (UC), irritable bowel syndrome (IBS), celiac disease and any combination thereof and any gut inflammation associated with one of the above listed inflammatory disorders.

As used herein, the term “spondyloarthritis” or abbreviated “SpA” refers to a group of closely related, but clinically heterogeneous, inflammatory arthritis diseases with common features, including inflammation of the spine, eyes, skin, joints and gastrointestinal tract. This SpA group is also sometimes referred to as spondylitis and spondyloarthropathies. As used herein, SpA includes ankylosing spondylitis (including non-radiographic axial SpA, i.e., ankylosing spondylitis diagnosed using MRI), reactive arthritis, psoriatic arthritis, enteropathic arthritis (arthritis associated with inflammatory bowel disease or IBD related arthritis), undifferentiated spondyloarthritis, juvenile idiopathic arthritis and juvenile-onset SpA. Characteristics of these SpA diseases include inflammatory arthritis of the spine, peripheral arthritis that differs from rheumatoid arthritis, extra articular manifestations of inflammatory bowel disease, arthritis and uveitis, seronegativity for rheumatoid factor and some degree of heritability, including the presence of the gene HLA-B27. It is thus clear that in current application SpA is not rheumatoid arthritis.

“Primary sclerosing cholangitis” or “PSC” as used herein refers to a severe chronic liver disease characterized by progressive biliary inflammation and fibrosis. The development of multifocal bile duct structures can lead to liver fibrosis and subsequent cirrhosis. Patients with PSC are usually asymptomatic and the diagnostic work up is triggered by incidental findings of altered liver enzymes. In symptomatic patients, fatigue, pruritus, abdominal pain and jaundice are the most reported symptoms (Lazaridis et al. 2016 N. Engl. J. Med. 375:1161-1170). Following clinical suspicion and a suggestive biochemistry, magnetic resonance cholangiography or endoscopic retrograde cholangiopancreatography are used to establish the diagnosis. Presently, liver biopsy is reserved to diagnose suspected small duct PSC or to exclude other diagnosis (Lindor et al. 2015 Am. J. Gastroenterol. 110:646-659). It would thus be highly advantageous to develop presymptomatic diagnostic methods or non-invasive diagnostic methods. The diagnostic methods disclosed above solve this technical problem. Therefore, in a particular embodiment, the herein disclosed methods are provided of diagnosing primary sclerosing cholangitis, more particularly gut inflammation associated with primary sclerosing cholangitis.

A systematic review of the epidemiologic studies in PSC reported an incidence varying between 0 and 1.3 cases per 100,000 individuals and a prevalence of 0-16.2 cases per 100,000 individuals (Boonstra et al. 2012 J. Hepatol. 56:1181-1188). Most commonly, PSC affects men at the age of 40 and the concomitant diagnose of IBD is very common. Between 60 to 80% of the patients with PSC have concomitantly IBD, most frequently UC, pointing toward the possible role of the colon in the pathogenesis of PSC (Boonstra et al. 2013 Hepatology 58:2045-2055). This role is further evidenced by transplantation data showing that colectomy before liver transplantation is a protective factor for recurrence of PSC after liver transplantation (Alabraba et al. 2009 Liver Transpl. 15:330-340). Interestingly, the absence of intestinal microbiota is associated with increased severity of the disease in mice models (Tabibian et al. 2016 Hepatology 63:185-196). Therefore, intestinal microbiota may play an important role in the pathogenesis of PSC by modulating the gut-associated immune system to a more immunogenic or tolerogenic phenotype. In patients with IBD, the prevalence of PSC varies from 0.4 to 6.4%. However, in a recent study using magnetic resonance to diagnose PSC in patients with IBD the prevalence of PSC was three-fold higher than previously reported, mainly due to subclinical PSC without symptoms or altered liver enzymes (Lunder et al. 2016 Gastroenterology 151:660-669).

Genome-wide association studies suggested a role for immune-related pathways in the pathogenesis of PSC. Patients with PSC have a higher activity of TH17 cells. These lymphocytes help in the defense against bacteria and fungi by promoting inflammation and are involved in autoimmune diseases (Katt et al. 2013 Hepatology 58:1084-1093). Moreover, Treg cells (CD4+CD25+FOXP3+CD127−), which suppress inflammation, are reduced in PSC (Sebode et al. 2014 J. Hepatol. 60:1010-1016). Therefore, in a very particular embodiment, the inflammatory disorder as mentioned in the application refers to inflammatory disorders characterized by a TH17 response.

“Multiple sclerosis” or “MS” as used herein refers to a chronic inflammatory and neurodegenerative disease characterized by substantial clinical heterogeneity. Both genetic and immunologic factors, as well as environmental elements contribute to its etiology. Most MS patients present with recurrent periods of relapses and remissions, with relapses thought to be provoked by the infiltration of adaptive immune cells into the central nervous system (CNS), hereby resulting in focal inflammation and myelin loss (Franciotta et al. 2008 Lancet neurology 7:852-588). In a minority of patients, slow progression is observed from onset. Therefore, three clinical phenotypes can be distinguished: relapsing-remitting (RR), secondary progressive (SP) or primary progressive (PP) MS. Lublin et al. (2014 Neurology 83:278-286) further described these phenotypes as active, not active, and with or without progression. While not recognized as a separate phenotype, a subset of RRMS patients appears to have a mild course, often referred to as benign MS (BMS) (Amato et al. 2006 J. Neurol. 253:1054-1059; Calabrese et al. 2013 Mult. Scler. 19:904-911). Patients experience a wide variety of symptoms, ranging from physical and cognitive symptoms to even bowel dysfunction, with the latter being reported in more than 70% of cases (Wiesel et al. 2001 Eur. J. Gastroenterol. Hepatol. 13:441-448). Studies in experimental allergic encephalomyelitis (EAE), a widely used mouse model for MS, have provided evidence for a substantial effect of gut microbiota on central nervous system (CNS)-specific autoimmune disease (Berer et al. 2014 FEBS letters 588:4207-4013). The absence of gut microbes (germ-free conditions) or the alteration of the gut microbial flora composition with antibiotics resulted in a shift in T cell responses (decreased concentration of IL-17, increased number of regulatory T and B cells) and affected disease severity (Ochoa-Reparaz et al. 2009 J. Immunol. 183:6041-6050). Additionally, mice raised in a germ-free environment were highly resistant to developing spontaneous EAE, unless exposed to specific pathogen-free condition-derived fecal material or a fecal transplant from MS twin-derived microbiota (Berer K. et al. 2011 Nature 479:538-541; Berer et al. 2017 Proc. Nat. Ac. Sc. USA). Immune cells from mouse recipients of MS-twin samples produced less IL-10 than immune cells from mice colonized with healthy-twin samples. IL-10 may have a regulatory role in spontaneous CNS autoimmunity, as neutralization of the cytokine in mice colonized with healthy-twin fecal samples increased disease incidence. This evidence suggests that the microbiota may be capable of altering the individual at a phenotypic level and influence the onset, severity and progression of MS. Therefore, in a particular embodiment, the methods disclosed herein are provided for detecting multiple sclerosis or gut inflammation associated with multiple sclerosis.

The wording “gut inflammation” is equivalent to the wording “microscopic gut inflammation” as used herein and refers to an inflammatory response in the gut as defined above. The inflammation can affect the entire gastrointestinal tract, can be more limited to, for example, the small intestine or large intestine but can also be limited to specific components or structures such as the bowel walls.

As used herein, the term “inflammatory bowel disease” or abbreviated “IBD” refers to an umbrella term for inflammatory conditions of the gut under which both Crohn's disease and ulcerative colitis fall. In people with IBD, the immune system mistakes food, bacteria, or other materials in the gut for foreign substances and responds by sending white blood cells into the lining of the bowels. The result of the immune system's attack is chronic inflammation. Crohn's disease and ulcerative colitis are the most common forms of IBD. Less common IBDs include microscopic colitis, diverticulosis-associated colitis, collagenous colitis, lymphocytic colitis and Behcet's disease. In the case of CD, transmural inflammation commonly affects the terminal ileum, although any part of the gastrointestinal system can be affected. Discontinuous inflammation and the presence of non-caveating granulomas are also characteristic of the inflammation in patients with CD. In contrast, UC is characterized by continuous mucosal inflammation starting in the rectum and extending proximally until the caecum (Harries et al. 1982 Br. Med. J. Clin. Res. Ed., 284:706). These are chronic relapsing diseases originating mostly during adolescence and young adulthood and are characterized by chronic inflammation of the gastrointestinal tract leading to invalidating symptoms of bloody diarrhea, weight loss and fatigue (Wilks 1859 Med. Times Gazette 2:264-265). Recent epidemiologic data from France reported a mean incidence of 4.4 cases per 100,000 individuals (Ghione et al. 2017 Am. J. Gastroenterol.). Worldwide, the incidence and prevalence of CD range from 0.0-29.3 per 100,000 person-years and 0.6-318.5 per 100,000 persons, respectively. The incidence and prevalence of UC varies from 0.0-19.2 per 100,000 person-years and 2.42-298.5 per 100,000 persons, respectively (Molodecky et al. 2012 Gastroenterology 142:46-54).

Several defects in innate and adaptive immunity have been described both in UC and CD (de Souza et al. 2016 Nat. Rev. Gastroenterol. Hepatol. 13:13-27). In normal conditions, intestinal macrophages exhibit inflammatory anergy, which allows the interaction with commensal flora without inducing strong inflammatory responses (Smythies et al. 2005 J. Clin. Invest. 115:66-75). However, CD14+ intestinal macrophages are more abundant in patients with CD than in healthy individuals. These CD14+ intestinal macrophages produce more proinflammatory cytokines, such as interleukin (IL)-6, IL-23 and tumor necrosis factor (TNF)-a, than the common CD14-intestinal macrophages (Kamada et al. 2008 J. Clin. Invest. 118:2269-2280). Adaptive immunity also plays a role in the pathogenesis of IBD. T helper (TH) lymphocytes are cytokine producing lymphocytes that potentiate or regulate immune responses by interacting with other immune cells such as macrophages, CD8+ T cells, eosinophils and basophils. Following an initial trigger (e.g., impaired barrier function by injury or exposure to xenobiotics) the microbe-associated molecular patterns will induce the secretion of cytokines by dendritic cells, epithelial cells and macrophages, among others. Different cytokine milieus will induce TH1, TH2, TH17 or regulatory T-cell (Treg) subsets (de Souza et al. 2016 Nat. Rev. Gastroenterol. Hepatol. 13:13-27). In susceptible individuals, an interplay between TH1 and TH17 immune responses seem to be linked with inflammation associated with CD. On the other hand, UC has been described as a TH2-like condition with possible implication of a newly discovered TH9 lymphocytes (de Souza et al. 2016 Nat. Rev. Gastroenterol. Hepatol. 13:13-27; Gerlach et al. 2014 Nat. Immunol. 15:676-686). In both diseases, an insufficient Treg response seems to be involved in the impaired regulation of inflammatory responses (Maul et al. 2005 Gastroenterology 128:1868-1878). In active IBD, the immune system shows an increased response to bacterial stimulation, thereby contributing even further to the chronic inflammatory state. This inflammatory state also produces an increase in the intestinal permeability, allowing bacterial antigens to contact with the immune system, hereby perpetuating the inflammatory state.

In particular embodiments, the inflammation or inflammatory disorder as used in the methods of the fifth aspect is inflammation or an inflammatory disorder characterized by a TH1, TH17, TH2 and/or TH9 response. In even more particular embodiments, the inflammation or inflammatory disorder is characterized by a TH1 and/or TH17 response.

The therapeutic options of the inflammatory disorder diagnosed using the methods herein provided comprise the commonly used anti-inflammatory drugs such as inhibitors of cyclooxygenase activity (aspirin, celecoxib, diclofenac, diflunisal, etodolac, ibuprofen, indomethacin, ketoprofen, ketorolac, meloxicam, nabumetone, naproxen, oxaprozin, piroxicam, salsalate, sulindac, tolmetin, among others) or corticosteroids (prednisone, dexamethasone, hydrocortisone, methylprednisolone, among others) or in combination with commonly used analgesics (acetaminophen, duloxetine, paracetamol, among others) or in any combination thereof. In particular embodiments, the anti-inflammatory therapy includes a biological therapy, such as TNF-alpha blockers, anti-IL17A monoclonal antibodies, anti-CD20 antibodies.

The therapeutic options for CD or UC include corticosteroids, aminosalicylates, immunosuppressive agents and biological therapies. Due to the chronic relapsing and remitting disease-course of IBD, the goal of medical therapy is to induce (induction phase) and maintain remission (maintenance phase). The choice between the different medical therapies depends on several factors such as disease location and severity, medical and surgical history, age, co-morbidities, extra-intestinal manifestations and treatment availability (Gomollon et al. 2017 J. Crohns Colitis 11:3-25; Harbord et al. 2017 J. Crohns Colitis 2017).

An “effective amount” of a composition is equivalent to the dosage of the composition that leads to treatment, prevention or a reduction of the severity of inflammation status in a patient. The inflammation can be gut inflammation for which several methods are known to the person skilled in the art to evaluate or thus to diagnose the severity of the inflammation.

Recently, Vieira-Silva et al. (2020 Nature 581: 310-315) reported that a higher prevalence of the B2 enterotype correlates with a higher body-mass index and obesity. Interestingly, the pattern of enterotypes found in the population of obese individuals differed significantly depending on whether people were taking cholesterol-lowering drugs called statins. Obese participants taking statins had a significantly lower prevalence of the B2 enterotype than did their obese counterparts not taking statins.

Therefore, methods of diagnosing and treating gut flora dysbiosis are provided. Methods of diagnosing a gut microbiome associated with or predictive for gut flora dysbiosis and/or inflammatory disorder and changing the gut microbiome to a healthy or non-disease associated gut flora are also provided. The methods comprise the steps from the methods provided in the fourth aspect of current application further comprising a step of administering an effective amount of a statin to the subject.

The methods comprise the following steps:
measuring in a biological sample of a subject the level of at least one metabolic biomarker selected from any of the metabolic biomarker panels of the application;
comparing the measured level of the at least one biomarker of the subject sample to that of a control sample;
treating the subject with an effective amount of a statin when the measured level of the at least one biomarker in the subject sample is increased or decreased relative to the level of the at least one biomarker in the control sample and/or if the difference between the measured level of the at least one biomarker in the subject sample and that of the control sample is statistically significant.

In one embodiment, the biological sample is selected from the list consisting of blood, serum and/or plasma.

Statins, also known as HMG-CoA reductase inhibitors, are a class of lipid-lowering medications that are often prescribed to reduce illness and mortality in those who are at high risk of cardiovascular disease. Statins are the most common cholesterol-lowering drugs. Non-limiting examples of statins are lovastatin, fluvastatin, pravastin, rosuvastatin, pitavastatin, atorvastatin, simvastatin, cerivastatin, mevastatin.

An “effective amount” of a statin is equivalent to the dosage of the statin that leads to change in gut microbiome in a subject. The change is a change from a B2 enterotype to a non-B2 enterotype or from a gut microbiome associated with gut flora dysbiosis and/or an inflammatory disorder to a healthy gut microbiome.

The following examples are intended to promote a further understanding of the disclosure. While the disclosure is described herein with reference to illustrated embodiments, it should be understood that the disclosure is not limited hereto. Those having ordinary skill in the art and access to the teachings herein will recognize additional modifications and embodiments within the scope thereof. Therefore, the disclosure is limited only by the claims attached herein.

### EXAMPLES

### Example 1: Selecting Blood Serum Metabolites to Predict the B2 Enterotype

To identify and characterize major gut microbiome-associated variables, the Flemish Gut Flora Project (FGFP) initiated a large-scale cross-sectional fecal sampling effort in a confined geographic region (Flanders, Belgium). FGFP collection protocols combined rigorous sampling logistics, including frozen sample collection and cold chain monitoring, with exhaustive phenotyping through online questionnaires, standardized anamnesis and health assessment by general medical practitioners (GPs), and extended clinical blood profiling. Encompassing an equilibrated range of age, gender, health, and lifestyle, the FGFP cohort is a representative for the average gut microbiota composition in a Western European population. From this cohort, blood serum metabolomics data and microbiome phylogenetic profiling based on 16S ribosomal RNA (rRNA) gene amplicon sequencing of stool samples was available for 2938 participants. In that dataset 1031 participants had a Bacteroides 1 type, 503 were determined to be Bacteroides 2, 900 participants a Ruminococcus and 504 a Prevotella type. To balance the dataset and to develop a method that distinguished B2 from non-B2 enterotypes, 500 participants with the B2 enterotype were randomly selected and 500 with another enterotype (i.e., B1, R or P). From these randomly selected participants, blood serum metabolites were analyzed. After removing uncharacterized metabolites, 1024 characterized metabolites were retained. Metabolite levels were centered around zero and the variation normalized using the StandardScaler implementation from scikit-learn.

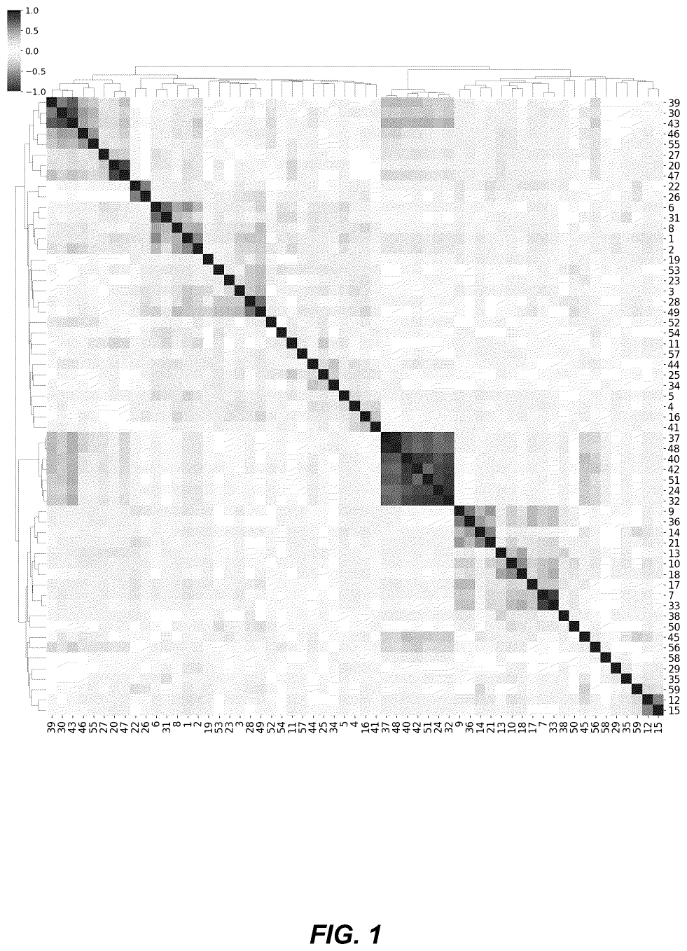

To filter the full list of metabolites to a more manageable set of metabolites for further analysis, feature selection (as statistical analysis tool) was used to pick up all metabolites with a significant variation between participants with a B2 enterotype and those with another enterotype after correcting for multiple testing using a Bonferroni test. The resulting set of 59 metabolites can be found in Table 1. As co-linear metabolites could be selected using this approach a correlation analysis was done to highlight (potential) dependencies between metabolites (FIG. 1).

TABLE 1
The full list of metabolites picked up using feature selection as highly relevant for predicting the B2
enterotype for blood serum metabolites. The column ″B2 level″ depicts whether the metabolite is
decreased or increased in subjects with a B2 enterotype. CAS, CAS number; HMDB, Human Metabolite
database identifier; round and square brackets with a single number indicate the metabolite
is a structural isomer.

| | | | p-value | | Fold change | | |
|---|---|---|---|---|---|---|---|
| METABOLITE | Score | p-value | (corrected) | B2 level | (log2) | CAS | HMDB |
| 3-phenylpropionate | 212.4 | 9.23E−44 | 9.45E−41 | decreased | −1.36 | 501-52-0 | HMDB00764 |
| (hydrocinnamate) | | | | | | | |
| cinnamoylglycine | 113.1 | 4.24E−25 | 4.34E−22 | decreased | −1.13 | 16534-24-0 | HMDB11621 |
| 5-hydroxyhexanoate | 98.8 | 2.92E−22 | 2.99E−19 | decreased | −0.37 | 44843-89-2 | HMDB00525 |
| 5alpha-androstan-3beta, | 87.0 | 6.80E−20 | 6.96E−17 | decreased | −1.02 | | |
| 17alpha-diol disulfate | | | | | | | |
| 4-hydroxycoumarin | 86.0 | 1.08E−19 | 1.11E−16 | decreased | −1.25 | 1076-38-6 | |
| hippurate | 81.8 | 7.66E−19 | 7.84E−16 | decreased | −0.75 | 495-69-2 | HMDB00714 |
| phenol sulfate | 71.4 | 1.04E−16 | 1.06E−13 | elevated | 0.87 | 937-34-8 | HMDB60015 |
| glucuronide of | 70.2 | 1.79E−16 | 1.83E−13 | decreased | −0.99 | | |
| C19H28O4 (2) | | | | | | | |
| isoursodeoxycholate | 69.6 | 2.42E−16 | 2.48E−13 | elevated | 1.75 | 78919-26-3 | HMDB00686 |
| imidazole propionate | 64.5 | 2.71E−15 | 2.77E−12 | elevated | 1.06 | 1074-59-5 | HMDB02271 |
| indolepropionylglycine | 58.5 | 4.84E−14 | 4.96E−11 | decreased | −0.93 | | |
| I-urobilinogen | 57.9 | 6.38E−14 | 6.53E−11 | elevated | 1.80 | 14684-37-8 | HMDB04157 |
| N-acetyl-cadaverine | 56.3 | 1.39E−13 | 1.42E−10 | elevated | 0.75 | 32343-73-0 | HMDB02284 |
| glycoursodeoxycholate | 45.6 | 2.41E−11 | 2.47E−08 | elevated | 1.11 | 64480-66-6 | HMDB00708 |
| D-urobilin | 45.1 | 3.16E−11 | 3.23E−08 | elevated | 1.08 | 3947-38-4 | HMDB04161 |
| 11-ketoetiocholanolone | 41.1 | 2.24E−10 | 2.30E−07 | decreased | −0.85 | 17181-16-7 | |
| glucuronide | | | | | | | |
| 7-alpha-hydroxy-3-oxo-4- | 40.9 | 2.48E−10 | 2.54E−07 | elevated | 0.27 | 115538-85-7 | HMDB12458 |
| cholestenoate (7-Hoca) | | | | | | | |
| glutarate (C5-DC) | 40.6 | 2.88E−10 | 2.94E−07 | elevated | 0.84 | 110-94-1 | HMDB00661 |
| 1H-indole-7-acetic acid | 39.4 | 5.05E−10 | 5.17E−07 | decreased | −0.82 | 39689-63-9 | |
| carotene diol (1) | 33.7 | 8.82E−09 | 9.03E−06 | decreased | −0.21 | | |
| ursodeoxycholate | 32.2 | 1.85E−08 | 1.89E−05 | elevated | 1.07 | 128-13-2 | HMDB00946 |
| taurolithocholate 3-sulfate | 31.7 | 2.29E−08 | 2.34E−05 | decreased | −0.48 | 64936-83-0 | HMDB02580 |
| indole-3-carboxylic acid | 28.8 | 9.99E−08 | 1.02E−04 | decreased | −0.27 | 771-50-6 | HMDB03320 |
| palmitoyl-linoleoyl- | 28.4 | 1.25E−07 | 1.28E−04 | elevated | 0.28 | | HMDB07103 |
| glycerol (16:0/18:2) [1] | | | | | | | |
| N2,N5-diacetylornithine | 27.5 | 1.91E−07 | 1.95E−04 | decreased | −0.30 | 39825-23-5 | |
| glycolithocholate sulfate | 26.7 | 2.92E−07 | 2.99E−04 | decreased | −0.41 | 15324-64-8 | HMDB02639 |
| beta-cryptoxanthin | 25.9 | 4.40E−07 | 4.51E−04 | decreased | −0.37 | 472-70-8 | HMDB33844 |
| phenylacetate | 25.6 | 5.02E−07 | 5.14E−04 | decreased | −0.40 | 103-82-2 | HMDB00209 |
| 3-(4-hydroxyphenyl) | 25.5 | 5.28E−07 | 5.41E−04 | elevated | 0.82 | 501-97-3 | HMDB02199 |
| propionate | | | | | | | |
| 1-(1-enyl-palmitoyl)-2- | 25.3 | 5.75E−07 | 5.89E−04 | decreased | −0.10 | | HMDB11206 |
| palmitoyl-GPC (P- | | | | | | | |
| 16:0/16:0) | | | | | | | |
| catechol sulfate | 25.2 | 6.16E−07 | 6.31E−04 | decreased | −0.33 | 4918-96-1 | HMDB59724 |
| palmitoyl-linoleoyl- | 24.0 | 1.12E−06 | 1.14E−03 | elevated | 0.31 | | HMDB07103 |
| glycerol (16:0/18:2) [2]* | | | | | | | |
| phenol glucuronide | 24.0 | 1.12E−06 | 1.15E−03 | elevated | 1.49 | 17685-05-1 | HMDB60014 |
| glucuronide of | 23.7 | 1.30E−06 | 1.33E−03 | decreased | −0.02 | | |
| C14H22O4 (2) | | | | | | | |
| dihydroferulic acid | 23.2 | 1.72E−06 | 1.76E−03 | elevated | 0.79 | 1135-23-5 | |
| N-acetylglucosamine | 22.7 | 2.20E−06 | 2.25E−03 | elevated | 1.68 | | |
| conjugate of C24H40O4 | | | | | | | |
| bile acid | | | | | | | |
| oleoyl-arachidonoyl- | 22.4 | 2.52E−06 | 2.58E−03 | elevated | 0.27 | | HMDB07228 |
| glycerol (18:1/20:4) [2] | | | | | | | |
| 4-hydroxyphenylacetate | 21.9 | 3.22E−06 | 3.29E−03 | elevated | 0.56 | 156-38-7 | HMDB00020 |
| 1-(1-enyl-palmitoyl)-2- | 21.8 | 3.49E−06 | 3.58E−03 | decreased | −0.11 | | HMDB11211 |
| linoleoyl-GPC (P- | | | | | | | |
| 16:0/18:2) | | | | | | | |
| oleoyl-oleoyl-glycerol | 21.7 | 3.68E−06 | 3.77E−03 | elevated | 0.31 | | HMDB07218 |
| (18:1/18:1) [2] | | | | | | | |
| etiocholanolone | 21.2 | 4.71E−06 | 4.82E−03 | decreased | −0.25 | 3602-09-3 | HMDB04484 |
| glucuronide | | | | | | | |
| palmitoyl-oleoyl-glycerol | 20.7 | 5.99E−06 | 6.13E−03 | elevated | 0.41 | | HMDB07102 |
| (16:0/18:1) [2] | | | | | | | |
| 1-(1-enyl-palmitoyl)-2- | 20.4 | 7.08E−06 | 7.26E−03 | decreased | −0.13 | | |
| oleoyl-GPC (P-16:0/18:1) | | | | | | | |
| 3-methyladipate | 20.3 | 7.48E−06 | 7.66E−03 | decreased | −0.29 | 3058-01-03 | HMDB00555 |
| 1-oleoyl-GPE (18:1) | 20.0 | 8.47E−06 | 8.68E−03 | elevated | 0.16 | 89576-29-4 | HMDB11506 |
| palmitoyl sphingomyelin | 20.0 | 8.67E−06 | 8.88E−03 | decreased | −0.04 | 6254-89-3 | |
| (d18:1/16:0) | | | | | | | |
| carotene diol (2) | 19.4 | 1.19E−05 | 1.21E−02 | decreased | −0.22 | | |
| oleoyl-arachidonoyl- | 19.2 | 1.29E−05 | 1.33E−02 | elevated | 0.26 | | HMDB07228 |
| glycerol (18:1/20:4) [1] | | | | | | | |
| p-cresol sulfate | 19.1 | 1.37E−05 | 1.41E−02 | decreased | −0.29 | 3233-57-7 | HMDB11635 |
| anthranilate | 18.9 | 1.49E−05 | 1.53E−02 | decreased | −0.14 | 118-92-3 | HMDB01123 |
| oleoyl-linoleoyl-glycerol | 18.7 | 1.67E−05 | 1.71E−02 | elevated | 0.20 | 104346-53-4 | HMDB07219 |
| (18:1/18:2) [2] | | | | | | | |
| guanidinosuccinate | 18.3 | 2.12E−05 | 2.17E−02 | decreased | −0.16 | 6133-30-8 | HMDB03157 |
| 5-hydroxyindole sulfate | 18.2 | 2.14E−05 | 2.19E−02 | decreased | −0.31 | | |
| 2-acetamidophenol sulfate | 18.0 | 2.46E−05 | 2.52E−02 | decreased | −0.60 | 40712-60-5 | |
| glycosyl-N-tricosanoyl- | 17.9 | 2.53E−05 | 2.59E−02 | decreased | −0.17 | | |
| sphingadienine | | | | | | | |
| (d18:2/23:0) | | | | | | | |
| 4-hydroxyglutamate | 17.4 | 3.32E−05 | 3.40E−02 | elevated | 0.26 | 2485-33-8 | HMDB01344 |
| 4-ethylphenylsulfate | 17.2 | 3.59E−05 | 3.68E−02 | decreased | −0.99 | 123-07-9 | |
| adenosine 5′- | 17.1 | 3.81E−05 | 3.90E−02 | decreased | −0.21 | 149022-20-8 | HMDB00045 |
| monophosphate (AMP) | | | | | | | |
| glycochenodeoxycholate | 16.7 | 4.75E−05 | 4.86E−02 | elevated | 0.41 | | |
| sulfate | | | | | | | |

In a next step, machine learning tools were used to determine if the selected 59 metabolites can be used to predict the B2 enterotype and what the impact is on the prediction of each of the 59 metabolites. Therefore, the dataset was split into a training set (90% of samples after balancing) and a testing set (the remaining 10%). First, different individual classifiers were trained on the training set. Because the obtained results depend on the specific classifier used, an ensemble classifier—which combines predictions made by multiple individual classifiers—was created as well to obtain more robust, higher quality results. In a second step, the test set was used to generate a classification report for the ensemble classifier as well as the individual classifiers it encapsulates.

The ensemble classifier resulted in a very good prediction of B2 versus non-B2 enterotype with a precision of 0.87, a recall of 0.87 and an F1-score of 0.87 (Table 2).

TABLE 2
Performance of the ensemble classifier
when predicting B2 vs non-B2

| | precision | recall | f1-score | support |
|---|---|---|---|---|
| B2 | 0.87 | 0.85 | 0.86 | 48 |
| non-B2 | 0.87 | 0.88 | 0.88 | 52 |
| accuracy | | | 0.87 | 100 |
| macro avg | 0.87 | 0.87 | 0.87 | 100 |
| weighted avg | 0.87 | 0.87 | 0.87 | 100 |

It outperformed all individual classifiers although the ADABoostClassifier performed very close to the ensemble classifier (Table 3). Since the ensemble classifier does not allow applying common evaluation metrics like the well-established ROC curves, the best individual classifier (i.e., the ADABoostClassifier), which does support evaluation metrics, was selected for the remaining parts of the application.

TABLE 3
Performance of the ADABoost Classifier
when predicting B2 vs non-B2

| | precision | recall | f1-score | support |
|---|---|---|---|---|
| B2 | 0.90 | 0.73 | 0.80 | 48 |
| non-B2 | 0.79 | 0.92 | 0.85 | 52 |
| accuracy | | | 0.83 | 100 |
| macro avg | 0.83 | 0.83 | 0.83 | 100 |
| weighted avg | 0.83 | 0.83 | 0.83 | 100 |

An additional advantage of using the ADABoostClassifier is that the most important features can be extracted from the classifier and ranked. This allows a further reduction of the number of required metabolites for the predictions. The complete list of metabolites and their individual impact on the decision process to distinguish the B2 enterotype from the others, for this particular classifier, is included in Table 4.

TABLE 4
Metabolites ranked by their individual impact on predicting the B2
enterotype using an ADABootsClassifier. The top ten (highlighted in grey),
was used in the next section.

| | Cumulative performance | | | | | |
|---|---|---|---|---|---|---|
| | | | Test Score | | ROC AUC | |
| METABOLITE | Importance | Count | (mean) | Std. | (mean) | Std. |
| 3-phenylpropionate | 0.05 | 1 | 0.761 | 0.037 | 0.759 | 0.005 |
| (hydrocinnamate) | | | | | | |
| isoursodeoxycholate | 0.05 | 2 | 0.773 | 0.046 | 0.734 | 0.017 |
| p-cresol sulfate | 0.05 | 3 | 0.760 | 0.067 | 0.779 | 0.010 |
| indole-3-carboxylic acid | 0.04 | 4 | 0.746 | 0.060 | 0.777 | 0.009 |
| glycolithocholate sulfate | 0.04 | 5 | 0.758 | 0.051 | 0.784 | 0.013 |
| 5-hydroxyhexanoate | 0.04 | 6 | 0.773 | 0.050 | 0.776 | 0.016 |
| anthranilate | 0.03 | 7 | 0.779 | 0.043 | 0.778 | 0.011 |
| adenosine 5′-monophosphate | 0.03 | 8 | 0.786 | 0.047 | 0.792 | 0.011 |
| (AMP) | | | | | | |
| glycoursodeoxycholate | 0.03 | 9 | 0.798 | 0.048 | 0.767 | 0.012 |
| glucuronide of C14H22O4 (2) | 0.03 | 10 | 0.798 | 0.040 | 0.816 | 0.016 |
| glutarate (C5-DC) | 0.03 | 11 | 0.807 | 0.047 | 0.806 | 0.012 |
| 1H-indole-7-acetic acid | 0.03 | 12 | 0.823 | 0.041 | 0.820 | 0.017 |
| guanidinosuccinate | 0.02 | 13 | 0.809 | 0.033 | 0.824 | 0.019 |
| oleoyl-linoleoyl-glycerol | 0.02 | 14 | 0.822 | 0.029 | 0.808 | 0.011 |
| (18:1/18:2) [2] | | | | | | |
| 4-ethylphenylsulfate | 0.02 | 15 | 0.817 | 0.033 | 0.817 | 0.012 |
| 1-oleoyl-GPE (18:1) | 0.02 | 16 | 0.822 | 0.038 | 0.802 | 0.023 |
| 1-(1-enyl-palmitoyl)-2-oleoyl- | 0.02 | 17 | 0.824 | 0.054 | 0.809 | 0.019 |
| GPC (P-16:0/18:1) | | | | | | |
| palmitoyl-oleoyl-glycerol | 0.02 | 18 | 0.828 | 0.051 | 0.808 | 0.015 |
| (16:0/18:1) [2] | | | | | | |
| catechol sulfate | 0.02 | 19 | 0.830 | 0.052 | 0.811 | 0.012 |
| cinnamoylglycine | 0.02 | 20 | 0.819 | 0.054 | 0.810 | 0.016 |
| palmitoyl sphingomyelin | 0.02 | 21 | 0.813 | 0.050 | 0.804 | 0.015 |
| (d18:1/16:0) | | | | | | |
| 1-(1-enyl-palmitoyl)-2- | 0.02 | 22 | 0.827 | 0.036 | 0.811 | 0.013 |
| palmitoyl-GPC (P-16:0/16:0) | | | | | | |
| 5alpha-androstan- | 0.02 | 23 | 0.834 | 0.032 | 0.812 | 0.011 |
| 3beta, 17alpha-diol disulfate | | | | | | |
| 4-hydroxycoumarin | 0.02 | 24 | 0.821 | 0.030 | 0.820 | 0.017 |
| hippurate | 0.02 | 25 | 0.822 | 0.034 | 0.815 | 0.016 |
| phenol sulfate | 0.02 | 26 | 0.804 | 0.042 | 0.813 | 0.019 |
| imidazole propionate | 0.02 | 27 | 0.821 | 0.040 | 0.823 | 0.017 |
| I-urobilinogen | 0.02 | 28 | 0.816 | 0.032 | 0.817 | 0.011 |
| N-acetyl-cadaverine | 0.02 | 29 | 0.823 | 0.041 | 0.825 | 0.010 |
| D-urobilin | 0.02 | 30 | 0.824 | 0.039 | 0.832 | 0.014 |
| taurolithocholate 3-sulfate | 0.01 | 31 | 0.838 | 0.037 | 0.827 | 0.016 |
| oleoyl-oleoyl-glycerol | 0.01 | 32 | 0.841 | 0.039 | 0.821 | 0.015 |
| (18:1/18:1) [2] | | | | | | |
| 4-hydroxyglutamate | 0.01 | 33 | 0.828 | 0.046 | 0.817 | 0.016 |
| 2-acetamidophenol sulfate | 0.01 | 34 | 0.834 | 0.042 | 0.826 | 0.014 |
| 5-hydroxyindole sulfate | 0.01 | 35 | 0.839 | 0.048 | 0.831 | 0.016 |
| glucuronide of C19H28O4 (2) | 0.01 | 36 | 0.826 | 0.031 | 0.831 | 0.018 |
| carotene diol (2) | 0.01 | 37 | 0.832 | 0.033 | 0.829 | 0.022 |
| indolepropionylglycine | 0.01 | 38 | 0.830 | 0.038 | 0.828 | 0.016 |
| etiocholanolone glucuronide | 0.01 | 39 | 0.831 | 0.039 | 0.835 | 0.017 |
| glycochenodeoxycholate sulfate | 0.01 | 40 | 0.828 | 0.029 | 0.833 | 0.013 |
| phenylacetate | 0.01 | 41 | 0.829 | 0.030 | 0.832 | 0.013 |
| 4-hydroxyphenylacetate | 0.01 | 42 | 0.828 | 0.036 | 0.850 | 0.013 |
| oleoyl-arachidonoyl-glycerol | 0.01 | 43 | 0.822 | 0.031 | 0.852 | 0.011 |
| N-acetylglucosamine conjugate | 0.01 | 44 | 0.836 | 0.035 | 0.856 | 0.013 |
| of C24H40O4 bile acid | | | | | | |
| dihydroferulic acid | 0.01 | 45 | 0.838 | 0.045 | 0.853 | 0.014 |
| ursodeoxycholate | 0.01 | 46 | 0.836 | 0.039 | 0.856 | 0.015 |
| phenol glucuronide | 0.01 | 47 | 0.833 | 0.036 | 0.856 | 0.016 |
| 7-alpha-hydroxy-3-oxo-4- | 0.01 | 48 | 0.829 | 0.039 | 0.849 | 0.021 |
| cholestenoate (7-Hoca) | | | | | | |
| carotene diol (1) | 0.01 | 49 | 0.824 | 0.033 | 0.855 | 0.012 |
| N2,N5-diacetylornithine | 0 | 50 | 0.828 | 0.034 | 0.854 | 0.018 |
| beta-cryptoxanthin | 0 | 51 | 0.828 | 0.034 | 0.854 | 0.018 |
| glycosyl-N-tricosanoyl- | 0 | 52 | 0.832 | 0.036 | 0.855 | 0.017 |
| sphingadienine (d18:2/23:0) | | | | | | |
| 1-(1-enyl-palmitoyl)-2- | 0 | 53 | 0.833 | 0.032 | 0.863 | 0.012 |
| linoleoyl-GPC (P-16:0/18:2) | | | | | | |
| 3-(4-hydroxyphenyl)propionate | 0 | 54 | 0.833 | 0.032 | 0.863 | 0.012 |
| palmitoyl-linoleoyl-glycerol | 0 | 55 | 0.824 | 0.027 | 0.865 | 0.011 |
| (16:0/18:2) [1] | | | | | | |
| oleoyl-arachidonoyl-glycerol | 0 | 56 | 0.824 | 0.027 | 0.865 | 0.011 |
| (18:1/20:4) [1] | | | | | | |
| 11-ketoetiocholanolone | 0 | 57 | 0.816 | 0.050 | 0.863 | 0.016 |
| glucuronide | | | | | | |
| 3-methyladipate | 0 | 58 | 0.816 | 0.047 | 0.863 | 0.020 |
| palmitoyl-linoleoyl-glycerol | 0 | 59 | 0.818 | 0.046 | 0.864 | 0.020 |
| (16:0/18:2) [2] | | | | | | |

To get an impression how robust the method is depending on the input data a more advanced cross validation scheme was used. Of the set of 500 B2 and 500 non-B2 samples, 100 samples were set aside for validation and creating the ROC curves, while a 10× cross-validation (CV) strategy was used on the remaining 900 samples. For each cross-validation iteration, a new model was trained on 90% of the set and tested against the remaining 10%. Each model from the CV along with the relevant metrics was stored. Using all 59 metabolites picked up using the feature selection the average test score is 0.818 (std. 0.046). A very similar results could be obtained when using only the top ten metabolites (using the feature impact, from the classifier described in Table 4), more precisely an average test score of 0.798 (std. 0.040) was obtained.

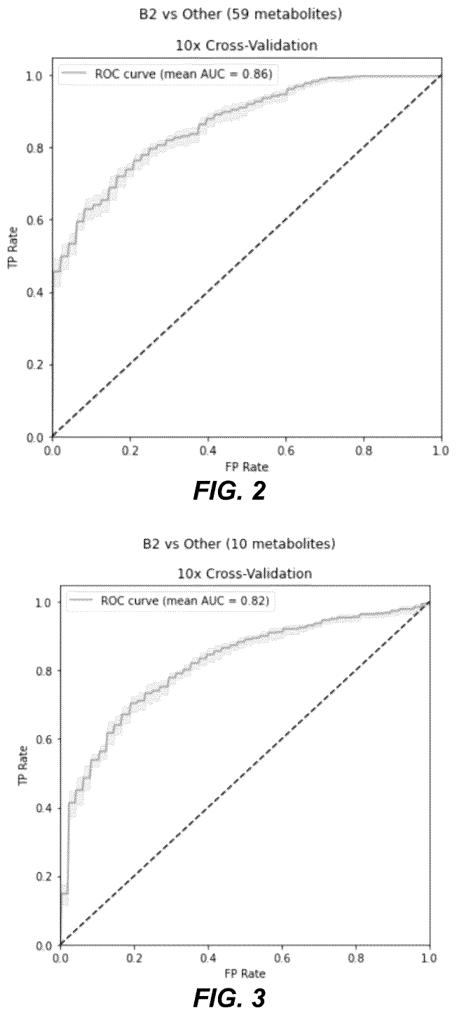

Next, ROC curves were generated using the cross-validation models and the 100 samples initially withheld. This allows the ROC curves to be drawn with a 95 percent confidence interval. When taking the full set of 59 metabolites into account (FIG. 2, mean area under the curve 0.86) or only taking into account the top ten metabolites (FIG. 3, mean area under the curve 0.82). While an expected drop in performance can be observed between using the full set and using only the top ten metabolites, classification methods with a ROC AUC >0.80 are considered excellent (Mandrekar 2010 J. Thorac. Oncol. 5: 1315-1316).

### Example 2

### Behavior of Selected Metabolites in the B2 Enterotype

For all the 59 metabolites selected above, the levels in the B2 and non-B2 enterotypes were inspected and checked for significant differences. Table 1 provides an overview of all selected metabolites and indicates if they are elevated of decreased in B2 compared to the non-B2 enterotypes (column “B2 level”).

### Example 3

### Defining the Minimal Number of Metabolites Required for B2 Prediction

Next, research was performed to determine whether the biomarker panel of ten metabolites could further be reduced and still provide an accurate B2 prediction. For this analysis, a classifier is considered acceptable if a ROC AUC of 0.7 or higher is obtained using the testing scheme outlined in the methods. To avoid excessive numbers of permutations to be necessary at later stages, a heuristic was used to estimate how many additional metabolites would likely be required for a given metabolite to yield acceptable predictions. To this end a classifier was generated using all metabolites and feature weights were determined as well as the cumulative performance using only the first, the first two, the first three, etc., metabolites (Table 4). The number of metabolites required to reach a ROC AUC of 0.7 was stored along with the required metabolites to reach that point, next the highest ranked feature was eliminated from the matrix and the same procedure was repeated until no metabolites remained in the list. For each metabolite, the smallest number of additional compounds required to reach a prediction with a ROC AUC>0.7 was determined. In the next step, where these findings are verified using random permutations with two, three or four metabolites, only metabolites that were likely to perform well in combination with one, two or three other metabolites were considered to reduce the number of required permutations and compute time.

By doing so, three classifiers were found based on individual metabolites, 1H-indole-7-acetic acid, 3-phenylpropionate (or hydrocinnamate) and cinnamoylglycine. All three metabolites are sufficiently decreased in the blood serum of participants with the B2 enterotype, compared to participants with another enterotype, that these can be used as a single marker with reasonable outcome of the predictions. Next, from metabolites where the heuristic indicated a combination of two metabolites would be sufficient, 5000 pairs were randomly selected, and tested. Metabolites in pairs that were able to generate classifiers with a ROC AUC >0.7 were selected. These thirteen metabolites were (glutarate (C5-DC), glycolithocholate sulfate, isoursodeoxycholate, 5alpha-androstan-3beta,17alpha-diol disulfate, hippurate, phenol sulfate, catechol sulfate, 4-hydroxycoumarin, I-urobilinogen, p-cresol sulfate, N-acetyl-cadaverine, imidazole propionate and 5-hydroxyhexanoate) confirmed to work in at least one pair. These twelve metabolites were removed from the matrix and 5000 triads with three metabolites selected by the heuristic to work in triple were created, models were made and their performance checked. This revealed that five metabolites (ursodeoxycholate, indole-3-carboxylic acid, phenol glucuronide, 7-alpha-hydroxy-3-oxo-4-cholestenoate (7-Hoca) and palmitoyl-oleoyl-glycerol (16:0/18:1) [2]) picked up by the initial heuristic all have potential combinations with three metabolites that generate acceptable predictions. The same method uncovered that combinations of four metabolites selected from a list of sixteen metabolites lead to an acceptable B2 prediction. The metabolites picked up at this step are: glucuronide of C14H22O4 (2), indolepropionylglycine, N-acetylglucosamine conjugate of C24H40O4 bile acid, glycoursodeoxycholate, phenylacetate, 4-ethylphenylsulfate, glycochenodeoxycholate sulfate, anthranilate, etiocholanolone glucuronide, 1-oleoyl-GPE (18:1), 4-hydroxyphenylacetate, dihydroferulic acid, 1-(1-enyl-palmitoyl)-2-oleoyl-GPC (P-16:0/18:1), adenosine 5′-monophosphate (AMP), 1-(1-enyl-palmitoyl)-2-palmitoyl-GPC (P-16:0/16:0) and palmitoyl sphingomyelin (d18:1/16:0).

### Materials and Methods

### Data Acquisition

Enterotypes for participants from the Flemish Gut Flora Project were determined using a Dirichlet Multinomial Model using the data and methodology described in Falony et al. (2016 Science). Blood serum metabolite levels were determined using liquid chromatography paired with a mass spectrometer (LC-MS). Unknown metabolites were removed prior to analysis. Metabolite levels were scaled using the StandardScaler. The dataset was balanced using random under-sampling to ensure an equal number of participants for each category were present in the final set.

### Feature Selection

The first selection of the most relevant metabolites to characterize the B2 enterotype, was done by computing the ANOVA F-value, p-value and corrected (Bonferroni) p-value for all metabolites and retaining those with a corrected p-value <0.05 (n =59). In practice, the f_classif function implemented in scikit-learn (version 0.23.1) was used.

### Classification Methods

An ensemble classifier (Voting Classifier) was created consisting of a DecisionTreeClassifier, a RandomForestClassifier (with 50 estimators), an AdaBoostClassifier (with 100 estimators), a Perceptron, a Support Vector Classifier, and a Stochastic Gradient Descent classifier. All with the default settings unless otherwise stated. All classifiers along with the VotingClassifier are implemented in scikit-learn.

### Method Evaluation

For testing the performance of individual classifiers the full dataset was split into a training and testing dataset in a 9/1 ratio. The ensemble classifier was trained on the training dataset and the performance (precision, recall and f1-scores) was for each individual classifier as well as the ensemble determined using the function classification report from scikit-learn.

### Cross Validation and ROC Curves

For further evaluation the dataset was converted to B2 and non-B2 enterotypes and balanced using random under-sampling. Using ten-fold cross validation, the performance was evaluated and ROC curves were created.

1.-16. (canceled)

17. A method comprising:
measuring a level of at least one metabolic biomarker selected from the group of metabolic biomarkers consisting of 3-phenylpropionate (hydrocinnamate), cinnamoylglycine, 5-hydroxyhexanoate, 5α-androstan-3β,17α-diol disulfate, 4-hydroxycoumarin, hippurate, phenol sulfate, glucuronide of C₁₉H₂₈O₄ (isoform 2), isoursodeoxycholate, imidazole propionate, indolepropionylglycine, I-urobilinogen, N-acetyl-cadaverine, glycoursodeoxycholate, D-urobilin, 11-ketoetiocholanolone glucuronide, 7-α-hydroxy-3-oxo-4-cholestenoate (7-Hoca), glutarate (C5-DC), 1H-indole-7-acetic acid, carotene diol (1), ursodeoxycholate, taurolithocholate 3-sulfate, indole-3-carboxylic acid, palmitoyl-linoleoyl-glycerol (16:0/18:2), N2,N5-diacetylornithine, glycolithocholate sulfate, β-cryptoxanthin, phenylacetate, 3-(4-hydroxyphenyl)propionate, 1-(1-enyl-palmitoyl)-2-palmitoyl-GPC (P-16:0/16:0), catechol sulfate, palmitoyl-linoleoyl-glycerol (16:0/18:2), phenol glucuronide, glucuronide of C₁₄H₂₂O₄ (isoform 2), dihydroferulic acid, N-acetylglucosamine conjugate of C₂₄H₄₀O₄ bile acid, oleoyl-arachidonoyl-glycerol (18:1/20:4) (isoform 2), 4-hydroxyphenylacetate, 1-(1-enyl-palmitoyl)-2-linoleoyl-GPC (P-16:0/18:2), oleoyl-oleoyl-glycerol (18:1/18:1) (isoform 2), etiocholanolone glucuronide, palmitoyl-oleoyl-glycerol (16:0/18:1) (isoform 2), 1-(1-enyl-palmitoyl)-2-oleoyl-GPC (P-16:0/18:1), 3-methyladipate, 1-oleoyl-GPE (18:1), palmitoyl sphingomyelin (d18:1/16:0), carotene diol (isoform 2), oleoyl-arachidonoyl-glycerol (18:1/20:4), p-cresol sulfate, anthranilate, oleoyl-linoleoyl-glycerol (18:1/18:2) (isoform 2), guanidinosuccinate, 5-hydroxyindole sulfate, 2-acetamidophenol sulfate, glycosyl-N-tricosanoyl-sphingadienine (d18:2/23:0), 4-hydroxyglutamate, 4-ethylphenylsulfate, adenosine 5′-monophosphate (AMP), and glycochenodeoxycholate sulfate in a biological sample, and
comparing the measured level of the at least one biomarker of the biological sample to a level of the at least one biomarker in a control sample.

18. The method according to claim 17, wherein the difference in the measured level of the at least one biomarker in the biological sample is statistically significantly different from that of the level of the at least one biomarker in the control sample.

19. The method according to claim 17, wherein the biological sample is selected from the group consisting of blood, serum, and plasma.

20. The method according to claim 17, further comprising diagnosing gut flora dysbiosis in the subject when the difference in the measured level of the at least one biomarker in the biological sample is statistically significantly different from that of the level of the at least one biomarker in the control sample.

21. The method according to claim 17, further comprising diagnosing a disease or disorder in the subject when the difference in the measured level of the at least one biomarker in the biological sample is statistically significantly different from that of the level of the at least one biomarker in the control sample.

22. The method according to claim 20, further comprising administering an effective amount of a statin to the subject.

23. The method according to claim 21, further comprising administering an effective amount of a statin to the subject.

24. The method according to claim 20, further comprising administering an effective amount of an anti-inflammatory drug to the subject.

25. The method according to claim 21, further comprising administering an effective amount of an anti-inflammatory drug to the subject.

26. A method comprising:
measuring a level of at least one metabolic biomarker selected from the group of metabolic biomarkers consisting of 1H-indole-7-acetic acid, 3-phenylpropionate, or cinnamoylglycine in a biological sample, and
comparing the measured level of the at least one biomarker of the biological sample to a level of the at least one biomarker in a control sample.

27. The method according to claim 26, further comprising:
measuring a level of at least one additional metabolic biomarker selected from the group of metabolic biomarkers consisting of 5-hydroxyhexanoate, 5α-androstan-3β,17α-diol disulfate, 4-hydroxycoumarin, hippurate, phenol sulfate, glucuronide of C₁₉H₂₈O₄ (isoform 2), isoursodeoxycholate, imidazole propionate, indolepropionylglycine, I-urobilinogen, N-acetyl-cadaverine, glycoursodeoxycholate, D-urobilin, 11-ketoetiocholanolone glucuronide, 7-α-hydroxy-3-oxo-4-cholestenoate (7-Hoca), glutarate (C5-DC), carotene diol (1), ursodeoxycholate, taurolithocholate 3-sulfate, indole-3-carboxylic acid, palmitoyl-linoleoyl-glycerol (16:0/18:2), N2,N5-diacetylornithine, glycolithocholate sulfate, β-cryptoxanthin, phenylacetate, 3-(4-hydroxyphenyl)propionate, 1-(1-enyl-palmitoyl)-2-palmitoyl-GPC (P-16:0/16:0), catechol sulfate, palmitoyl-linoleoyl-glycerol (16:0/18:2), phenol glucuronide, glucuronide of C₁₄H₂₂O₄ (isoform 2), dihydroferulic acid, N-acetylglucosamine conjugate of C₂₄H₄₀O₄ bile acid, oleoyl-arachidonoyl-glycerol (18:1/20:4) (isoform 2), 4-hydroxyphenyl acetate, 1-(1-enyl-palmitoyl)-2-linoleoyl-GPC (P-16:0/18:2), oleoyl-oleoyl-glycerol (18:1/18:1) (isoform 2), etiocholanolone glucuronide, palmitoyl-oleoyl-glycerol (16:0/18:1) (isoform 2), 1-(1-enyl-palmitoyl)-2-oleoyl-GPC (P-16:0/18:1), 3-methyladipate, 1-oleoyl-GPE (18:1), palmitoyl sphingomyelin (d18:1/16:0), carotene diol (isoform 2), oleoyl-arachidonoyl-glycerol (18:1/20:4), p-cresol sulfate, anthranilate, oleoyl-linoleoyl-glycerol (18:1/18:2) (isoform 2), guanidinosuccinate, 5-hydroxyindole sulfate, 2-acetamidophenol sulfate, glycosyl-N-tricosanoyl-sphingadienine (d18:2/23:0), 4-hydroxyglutamate, 4-ethylphenylsulfate, adenosine 5′-monophosphate (AMP), and glycochenodeoxycholate sulfate in a biological sample, and
comparing the measured level of the at least one additional biomarker of the biological sample to a level of the at least one additional biomarker in a control sample.

28. The method according to claim 26, wherein the difference in the measured level of the at least one biomarker in the biological sample is statistically significantly different from that of the level of the at least one biomarker in the control sample.

29. The method according to claim 26, wherein the biological sample is selected from the group consisting of blood, serum, and plasma.

30. The method according to claim 26, further comprising diagnosing gut flora dysbiosis in the subject when the difference in the measured level of the at least one biomarker in the biological sample is statistically significantly different from that of the level of the at least one biomarker in the control sample.

31. The method according to claim 26, further comprising diagnosing a disease or disorder in the subject when the difference in the measured level of the at least one biomarker in the biological sample is statistically significantly different from that of the level of the at least one biomarker in the control sample.

32. The method according to claim 30, further comprising administering an effective amount of a statin to the subject.

33. The method according to claim 30, further comprising administering an effective amount of an anti-inflammatory drug to the subject.

34. The method according to claim 31, further comprising administering an effective amount of a statin to the subject.

35. The method according to claim 31, further comprising administering an effective amount of an anti-inflammatory drug to the subject.

36. A method of diagnosing a subject for gut flora dysbiosis, an inflammatory disorder, obesity, diabetes type 2, and/or depression, the method comprising:
measuring a level of at least one metabolic biomarker selected from the group of metabolic biomarkers consisting of 3-phenylpropionate (hydrocinnamate), cinnamoylglycine, 5-hydroxyhexanoate, 5α-androstan-3β,17α-diol disulfate, 4-hydroxycoumarin, hippurate, phenol sulfate, glucuronide of C₁₉H₂₈O₄ (isoform 2), isoursodeoxycholate, imidazole propionate, indolepropionylglycine, I-urobilinogen, N-acetyl-cadaverine, glycoursodeoxycholate, D-urobilin, 11-ketoetiocholanolone glucuronide, 7-α-hydroxy-3-oxo-4-cholestenoate (7-Hoca), glutarate (C5-DC), 1H-indole-7-acetic acid, carotene diol (1), ursodeoxycholate, taurolithocholate 3-sulfate, indole-3-carboxylic acid, palmitoyl-linoleoyl-glycerol (16:0/18:2), N2,N5-diacetylornithine, glycolithocholate sulfate, β-cryptoxanthin, phenylacetate, 3-(4-hydroxyphenyl)propionate, 1-(1-enyl-palmitoyl)-2-palmitoyl-GPC (P-16:0/16:0), catechol sulfate, palmitoyl-linoleoyl-glycerol (16:0/18:2), phenol glucuronide, glucuronide of C₁₄H₂₂O₄ (isoform 2), dihydroferulic acid, N-acetylglucosamine conjugate of C₂₄H₄₀O₄ bile acid, oleoyl-arachidonoyl-glycerol (18:1/20:4) (isoform 2), 4-hydroxyphenylacetate, 1-(1-enyl-palmitoyl)-2-linoleoyl-GPC (P-16:0/18:2), oleoyl-oleoyl-glycerol (18:1/18:1) (isoform 2), etiocholanolone glucuronide, palmitoyl-oleoyl-glycerol (16:0/18:1) (isoform 2), 1-(1-enyl-palmitoyl)-2-oleoyl-GPC (P-16:0/18:1), 3-methyladipate, 1-oleoyl-GPE (18:1), palmitoyl sphingomyelin (d18:1/16:0), carotene diol (isoform 2), oleoyl-arachidonoyl-glycerol (18:1/20:4), p-cresol sulfate, anthranilate, oleoyl-linoleoyl-glycerol (18:1/18:2) (isoform 2), guanidinosuccinate, 5-hydroxyindole sulfate, 2-acetamidophenol sulfate, glycosyl-N-tricosanoyl-sphingadienine (d18:2/23:0), 4-hydroxyglutamate, 4-ethylphenylsulfate, adenosine 5′-monophosphate (AMP), and glycochenodeoxycholate sulfate in a biological sample, and
comparing the measured level of the at least one biomarker of the biological sample to a level of the at least one biomarker in a control sample, so as to diagnose gut flora dysbiosis, an inflammatory disorder, obesity, diabetes type 2, or depression in the subject.

37. The method according to claim 17, wherein
at least one biomarker is selected from the group consisting of 1H-indole-7-acetic acid, 3-phenylpropionate, and cinnamoylglycine;
at least two biomarkers are selected from the group consisting of imidazole propionate, 4-hydroxycoumarin, catechol sulfate, glycolithocholate sulfate, I-urobilinogen, phenol sulfate, isoursodeoxycholate, p-cresol sulfate, hippurate, hydroxyhexanoate, N-acetyl-cadaverine, glutarate (C5-DC), 5α-androstan-3β, and 17α-diol disulfate;
at least three biomarkers are selected from the group consisting of ursodeoxycholate,7-α-hydroxy-3-oxo-4-cholestenoate (7-Hoca), indole-3-carboxylic acid, palmitoyl-oleoyl-glycerol (16:0/18:1) (isoform 2), and phenol glucuronide;
at least four biomarkers are selected from the group consisting of glucuronide of C₁₄H₂₂O₄ (isoform 2), etiocholanolone glucuronide, indolepropionylglycine, 1-oleoyl-GPE (18:1), N-acetylglucosamine conjugate of C₂₄H₄₀O₄ bile acid, 4-hydroxyphenylacetate, glycoursodeoxycholate, dihydroferulic acid, phenylacetate, 1-(1-enyl-palmitoyl)-2-oleoyl-GPC (P-16:0/18:1), 4-ethylphenyl sulfate, AMP, glycochenodeoxycholate sulfate, 1-(1-enyl-palmitoyl)-2-palmitoyl-GPC (P-16:0/16:0), anthranilate, and palmitoyl sphingomyelin (d18:1/16:0));
at least six biomarkers are selected from the group consisting of p-cresol sulfate, 3-phenylpropionate, anthranilate, 4-hydroxycoumarin, glutarate (C5-DC), indole-3-carboxylic acid, glycolithocholate sulfate, isoursodeoxycholate, 1H-indole-7-acetic acid, glycoursodeoxycholate, glucuronide of C₁₄H₂₂O₄ (2), 5-hydroxyhexanoate, 5α-androstan-3β,17α-diol disulfate, 1-(1-enyl-palmitoyl)-2-palmitoyl-GPC (P-16:0/16:0), and adenosine 5′-monophosphate (AMP); or at least eleven biomarkers are selected from the group consisting of catechol sulfate, 1-oleoyl-GPE (18:1), phenylacetate, cinnamoylglycine, carotene diol (2), ursodeoxycholate, 2-acetamidophenol sulfate, 5-hydroxyindole sulfate, etiocholanolone glucuronide, dihydroferulic acid, phenol glucuronide, glucuronide of C₁₉H₂₈O₄ (2), glycochenodeoxycholate sulfate, indolepropionylglycine, 4-hydroxyphenylacetate, palmitoyl sphingomyelin (d18:1/16:0), oleoyl-arachidonoyl-glycerol (18:1/20:4) (isoform 2), palmitoyl-oleoyl-glycerol (16:0/18:1) (isoform 2), N-acetylglucosamine conjugate of C₂₄H₄₀O₄ bile acid, 1-(1-enyl-palmitoyl)-2-oleoyl-GPC (P-16:0/18:1), and 7-α-hydroxy-3-oxo-4-cholestenoate (7-Hoca).
